SEQUENCE LISTING (40 sequences)

SEQ ID NO: 1 - protein, 10 aa, synthetic construct
ILWQRRQRRG

SEQ ID NO: 2 - protein, 16 aa, synthetic construct
ILWQRRQRRGEERKAP

SEQ ID NO: 3 - protein, 16 aa, synthetic construct
LLWQRRQRRGEERKAP

SEQ ID NO: 4 - protein, 16 aa, synthetic construct
VLWQRRQRRGEERKAP

SEQ ID NO: 5 - protein, 10 aa, synthetic construct
LLWQRRQRRG

SEQ ID NO: 6 - protein, 10 aa, synthetic construct
VLWQRRQRRG

SEQ ID NO: 7 - protein, 16 aa, synthetic construct
IIWQRRQRRGEERKAP

SEQ ID NO: 8 - protein, 16 aa, synthetic construct
IVWQRRQRRGEERKAP

SEQ ID NO: 9 - protein, 10 aa, synthetic construct
IIWQRRQRRG

SEQ ID NO: 10 - protein, 10 aa, synthetic construct
IVWQRRQRRG

SEQ ID NO: 11 - protein, 16 aa, synthetic construct
ILFQRRQRRGEERKAP

SEQ ID NO: 12 - protein, 16 aa, synthetic construct
ILYQRRQRRGEERKAP

SEQ ID NO: 13 - protein, 10 aa, synthetic construct
ILFQRRQRRG

SEQ ID NO: 14 - protein, 10 aa, synthetic construct
ILYQRRQRRG

SEQ ID NO: 15 - protein, 16 aa, synthetic construct
ILWNRRQRRGEERKAP

SEQ ID NO: 16 - protein, 16 aa, synthetic construct
ILWQRRNRRGEERKAP

SEQ ID NO: 17 - protein, 10 aa, synthetic construct
ILWNRRQRRG

SEQ ID NO: 18 - protein, 10 aa, synthetic construct
ILWQRRNRRG

SEQ ID NO: 19 - protein, 16 aa, synthetic construct
ILWQKRQRRGEERKAP

SEQ ID NO: 20 - protein, 16 aa, synthetic construct
ILWQRKQRRGEERKAP

SEQ ID NO: 21 - protein, 16 aa, synthetic construct
ILWQRRQKRGEERKAP

SEQ ID NO: 22 - protein, 16 aa, synthetic construct
ILWQRRQRKGEERKAP

SEQ ID NO: 23 - protein, 16 aa, synthetic construct
ILWQRRQRRGEEKKAP

SEQ ID NO: 24 - protein, 10 aa, synthetic construct
ILWQKRQRRG

SEQ ID NO: 25 - protein, 10 aa, synthetic construct
ILWQRKQRRG

SEQ ID NO: 26 - protein, 10 aa, synthetic construct
ILWQRRQKRG

SEQ ID NO: 27 - protein, 10 aa, synthetic construct
ILWQRRQRKG

SEQ ID NO: 28 - protein, 16 aa, synthetic construct
ILWQRRQRRAEERKAP

SEQ ID NO: 29 - protein, 10 aa, synthetic construct
ILWQRRQRRA

SEQ ID NO: 30 - protein, 16 aa, synthetic construct
ILWQRRQRRGDERKAP

SEQ ID NO: 31 - protein, 16 aa, synthetic construct
ILWQRRQRRGEDRKAP

SEQ ID NO: 32 - protein, 16 aa, synthetic construct
ILWQRRQRRGEERRAP

SEQ ID NO: 33 - protein, 16 aa, synthetic construct
ILWQRRQRRGEERKGP

SEQ ID NO: 34 - protein, 16 aa, synthetic construct
  SITE 1: note = D amino acid
  SITE 2: note = D amino acid
ILWQRRQRRGEERKAH

SEQ ID NO: 35 - protein, 14 aa, synthetic construct
RRQRRGEERKAPGP

SEQ ID NO: 36 - protein, 10 aa, synthetic construct
ILWQRRQRRG

SEQ ID NO: 37 - protein, 13 aa, synthetic construct
  SITE 2: note = D amino acid
  SITE 3: note = D amino acid
PGPILWQRRQRRG

SEQ ID NO: 38 - protein, 10 aa, synthetic construct
  SITE 1: note = D amino acid
  SITE 2: note = D amino acid
LLWQRRQRRG

SEQ ID NO: 39 - protein, 12 aa, synthetic construct
GRKKRRQRRRPQ

SEQ ID NO: 40 - protein, 12 aa, synthetic construct
RRQRRGEERKAP

(19) United States
(12) Patent Application Publication
(10) Pub. No.: US 2024/0390455 A1
(43) Pub. Date: Nov. 28, 2024

(54) METHODS AND COMPOSITIONS FOR TREATING CANCERS AND OTHER PROLIFERATIVE DISORDERS

(71) Applicant: ARIZONA BOARD OF REGENTS ON BEHALF OF THE UNIVERSITY OF ARIZONA, Tucson, AZ (US)

(72) Inventors: Olga Rafikova, Tucson, AZ (US); Ruslan Rafikov, Tucson, AZ (US)

(21) Appl. No.: 18/797,990

(22) Filed: Aug. 8, 2024

Related U.S. Application Data
(63) Continuation-in-part of application No. PCT/US23/14131, filed on Feb. 28, 2023.
(60) Provisional application No. 63/314,883, filed on Feb. 28, 2022.

(51) Int. Cl. A61K 38/10; A61K 38/08; A61P 9/12

(52) U.S. Cl. CPC A61K 38/10; A61K 38/08; A61P 9/12

(57) ABSTRACT

Inositol monophosphatase 1 (IMPA1) controls the activity of the primary cell proliferation signaling cascades activated through the inositol-dependent phosphorylation of Akt and PKCs. Furthermore, IMPA1-mediated control of cell proliferation involves the recruitment of cytosolic IMPA1 on a plasma membrane by the receptor for advanced glycation endproducts (RAGE). Interaction between IMPA1 and RAGE redistributes inositols from the cytosolic pool to membrane microdomains and amplifies the synthesis of membrane-bound phosphatidylinositols (PtdIns) to trigger inositol-dependent cell proliferation. Thus, the peptide compositions described herein inhibit the formation of the complex between IMPA1 and RAGE (e.g., a pathogenic complex) to control over-proliferative cells.

### CROSS-REFERENCES TO RELATED APPLICATIONS

This application is a continuation-in-part and claims benefit of PCT Patent Application No. PCT/US23/14131 filed Feb. 28, 2023, which claims benefit of U.S. Provisional Application No. 63/314,883 filed Feb. 28, 2022, the specifications of which are incorporated herein in their entirety by reference.

### STATEMENT REGARDING FEDERALLY SPONSORED RESEARCH OR DEVELOPMENT

This invention was made with government support under Grant No. HL133085 and HL132918, awarded by National Institutes of Health. The government has certain rights in the invention.

### REFERENCE TO AN ELECTRONIC SEQUENCE LISTING

The contents of the electronic sequence listing (ARIZ 22_05 PCT_CIP.xml; Size: 38,145 bytes; and Date of Creation: Jul. 26, 2024) is herein incorporated by reference in its entirety.

### FIELD OF THE INVENTION

The present invention features peptides targeting inositols mediated proliferative signaling for treating cancers and other proliferative disorders.

### BACKGROUND OF THE INVENTION

Pulmonary arterial hypertension (PAH) is a complex disease defined as a progressive increase in pulmonary vascular resistance that leads to right ventricle overload, dysfunction, and failure. The obliteration of small pulmonary arteries due to the thickening of intimal and medial layers and the formation of angioproliferative lesions is the major contributor to the increased pulmonary pressure. Nevertheless, the current therapies predominantly target pulmonary vasoconstriction rather than vascular remodeling.

The insufficiency of the current PAH therapies is related to the complex nature of pathological events found at the stage of the developed disease. These include impaired cross-talk between the dysfunctional endothelial cells and other components of the pulmonary vascular wall, dysregulated cellular energetics and metabolism, altered cell survival mechanisms, and activation of inflammatory and immune pathways. Therefore, evaluating the specific mechanisms responsible for the early transition of normal vascular cells to highly proliferative cells seems critical to developing effective treatment approaches. Unfortunately, diagnosis of PAH at the early stage is complicated due to the nonspecific nature of PAH symptoms. This limitation, coupled with the insufficiency of the current screening tests to identify early PAH, prevents the scientific community from having a clear understanding of the pathways that predetermine PAH development.

### BRIEF SUMMARY OF THE INVENTION

It is an objective of the present invention to provide compositions and methods that allow for targeting inositol mediated proliferative signaling for treating cancers and other proliferative disorders, as specified in the independent claims. Embodiments of the invention are given in the dependent claims. Embodiments of the present invention can be freely combined with each other if they are not mutually exclusive.

Inositol monophosphatase 1 (IMPA1) is a rate-controlling enzyme in the synthesis of myo-inositol, the primary precursor for all phosphoinositides and inositol phosphates. The Inventors discovered that IMPA1 controls the activity of the primary cell proliferation signaling cascades activated through the inositol-dependent phosphorylation of Akt and PKCs. Furthermore, IMPA1-mediated control of cell proliferation involves the recruitment of cytosolic IMPA1 on a plasma membrane by the receptor for advanced glycation endproducts (RAGE). Interaction between IMPA1 and RAGE redistributes inositols from the cytosolic pool to membrane microdomains and amplifies the synthesis of membrane-bound phosphatidylinositols (PtdIns) to trigger inositol-dependent cell proliferation.

In some aspects, the present invention describes peptide inhibitors designed to block the interaction between IMPA1 and RAGE and prevent the activation of the pathological proliferative signal. The peptides were designed using computational modeling that predicted the RAGE/IMPA1 interacting interface and reproduced the part of RAGE involved in this interaction. By binding to IMPA1, these peptides prevent interaction between IMPA1 and full-size RAGE and inhibit the downstream proliferative signaling. The arrest of cell growth in the presence of inhibitory peptides was confirmed in human cancer cells and cancer-like over-proliferative pulmonary vascular cells isolated from patients with pulmonary arterial hypertension (PAH). Since the IMPA1/RAGE complex does not form in healthy cells, the proliferation rate of healthy cells in the presence of peptide inhibitors remains intact. Thus, the newly designed peptide inhibitors represent a unique therapeutic strategy that can selectively and efficiently arrest the growth of over-proliferative cells in patients with cancers and other proliferative disorders without producing any toxicity in the healthy cell population.

In some embodiments, the present invention features a peptide comprising a sequence at least 80% identical to ILWQRRQRRG (SEQ ID NO: 1). In other embodiments, the present invention features a peptide comprising a sequence at least 80% identical to ILWQRRQRRGEERKAP (SEQ ID NO: 2).

One of the unique and inventive technical features of the present invention is the compositions and methods that control cellular proliferation described herein. Without wishing to limit the invention to any theory or mechanism, it is believed that the technical feature of the present invention advantageously provides for only disrupting pathogenic protein-protein interactions without affecting normal protein function. Therefore, instead of inducing cell death, it restores a healthy proliferative rate. None of the presently known prior references or work has the unique, inventive technical feature of the present invention.

In fact, the prior references teach away from the present invention. For example, current techniques cannot target specific protein-protein interactions to reduce cellular proliferation and instead also target normal protein interactions. However, the present invention has discovered that the activity of the entire growth pathway depends on protein-protein interaction. The protein of interest (IMPA1) should be bound to the membrane receptor (RAGE) to overstimulate proliferative pathways occurring on the membrane microdomains. Without this interaction, the cytosolic IMPA1 does not induce the overstimulation of the growth (which is happening in healthy cells). Therefore, the regular approach of using small molecule inhibitors will non-selectively inhibit normal (cytosolic) and pathological (membrane) activity of IMPA1 and will be toxic for healthy cells. The peptide-based inhibitor that prevents RAGE/IMPA1 interaction will affect only over-proliferative cells.

Furthermore, the inventive technical feature of the present invention contributed to a surprising result. For example, the peptides described herein do not affect healthy cells. Additionally, the peptides described herein do not affect the catalytic activity of either IMPA1 or RAGE. The ability of the peptides described herein to selectively prevent interaction between two proteins without altering their activity 1) confirms the critical importance of this interaction for cell growth and 2) provides the opportunity for a targeted therapeutic approach.

Any feature or combination of features described herein are included within the scope of the present invention provided that the features included in any such combination are not mutually inconsistent as will be apparent from the context, this specification, and the knowledge of one of ordinary skill in the art. Additional advantages and aspects of the present invention are apparent in the following detailed description and claims.

### BRIEF DESCRIPTION OF THE SEVERAL VIEWS OF THE DRAWING(S)

The features and advantages of the present invention will become apparent from a consideration of the following detailed description presented in connection with the accompanying drawings in which:

FIGS. 1A, 1B, 1C, and 1D show an angioproliferative model of pulmonary arterial hypertension (PAH) induces severe changes in right ventricle (RV) pressure, RV hypertrophy, and RV function. Injection of Sprague-Dawley female rats with SU5416 followed by 3 weeks of hypoxia and 2 weeks of normoxia induced a progressive increase of right ventricle pressure (FIG. 1A); RV hypertrophy was measured as a wet weight ratio of the RV free wall normalized on the left ventricle-Fulton index (FIG. 1B), and changes in RV contractility (FIG. 1C) and RV relaxation (FIG. 1D) evaluated by measuring RV maximal (dP/dt) and minimal rate of RV pressure (dP/dt). PAH progression was especially evident at the early stage (e.g., week 1) and late-stage (e.g., week 5) of PAH. Results are expressed as box whisker plots (boxes: 25-75% percentile of the data; whiskers: minimum to maximum; the line represents the median value); n=8 rats in each group. * P<0.05 vs. control group; #P<0.05 vs. week 1; †P<0.05 vs. week 2. Statistical analysis was performed by Newman-Keuls Multiple-comparisons test. RVSP, right ventricle systolic pressure; RV/LV+S, right ventricle/left ventricle plus septum ratio.

FIGS. 2A and 2B show small pulmonary arteries become progressively remodeled in pulmonary arterial hypertension (PAH). FIG. 2A shows representative images from hematoxylin and eosin-stained pulmonary arteries (PA) of control and PAH rats at different stages of the disease. FIG. 2B shows a quantitative analysis of the vascular wall thickness. Twenty random PA per animal were analyzed. Vascular thickness was significantly higher in PAH rats compared with controls in both categories of PA examined (<150 and ≥150 μm). The remodeling of the smaller vessels progressed throughout the study, whereas hypertrophy of larger vessels showed an early increase and then stayed preserved. Results are expressed as box whisker plots (boxes: 25-75% percentile of the data; whiskers: minimum to maximum; the line represents the median value); n=8 rats for control and week 1 groups, n=7 for week 2 group, and n=6 for week 5 group. *P<0.05 vs. control group; #P<0.05 vs. week 1 group. Statistical analysis was performed by Newman-Keuls multiple-comparisons test. Open bars correspond to 100 μm.

FIGS. 3A, 3B, and 3C show pulmonary apoptosis and receptor for advanced glycation endproduct (RAGE) activation occur in the early and late stages of pulmonary arterial hypertension (PAH). The development of PAH was associated with early (e.g., week 1) and late (e.g., week 5) episodes of apoptosis in pulmonary tissue and pulmonary vascular wall. FIG. 3A shows total pulmonary apoptosis evaluated at different time points of PAH progression by measuring the levels of the proapoptotic marker cleaved caspase 3 in total lung lysate. The protein loading was normalized per total sample protein using stain-free imaging technology; n=5 rats in each group. FIG. 3B shows the level of apoptosis in the pulmonary artery vascular wall visualized by terminal deoxynucleotidyl transferase-mediated dUTP nick-end labeling (TUNEL). Images represent 6 to 10 pulmonary arteries per rat with n=4 rats/group. Black arrowheads, TUNEL-positive endothelial cells; open arrowheads, TUNEL-positive smooth muscle cells; black arrows, TUNEL-positive adventitial cells. The black bar corresponds to 100 μm. FIG. 3C shows the increase in pulmonary vascular apoptosis correlated with RAGE activation assessed by measuring the level of RAGE interaction with its adaptor protein myeloid differentiation primary response 88 (MyD88; C); n=4 rats in each group. Graphs are expressed as box whisker plots (boxes: 25-75th percentile of the data; whiskers: minimum to maximum; the line represents the median value). *P<0.05 vs. control group. Statistical analysis was performed by Newman-Keuls multiple-comparisons test. Significance between control and week 5 was confirmed using an unpaired t-test. RAGE indicates advanced glycation end products

FIGS. 4A, 4B, and 4C show a receptor for advanced glycation endproducts (RAGE) interacting with inositol monophosphatase 1 (IMPA1) in pulmonary arterial hypertension (PAH). Mass spectrometry analysis of proteins co-immunoprecipitated (co-IP) with RAGE from lungs identified IMPA1 as a novel RAGE interacting partner. The RAGE-IMPA1 complex was discovered in both samples from PAH animals (week 5) but not in control (peptide probability P>0; 4 peptides with P>89%). FIG. 4A shows a time course of RAGE-IMPA1 interaction was investigated by co-IP. There was no interaction between RAGE and IMPA1 in controls from week 1 RAGE/IMPA1 were efficiently co-IP (n=4 for all groups). Results are expressed as box and whisker plots (boxes: 25th to 75th percentile of the data; whiskers: minimum to maximum; the line represents the median value). *P<0.05 vs. control. Statistical analysis was performed by Newman-Keuls multiple-comparisons test. FIG. 4B shows the formation of the RAGE-IMPA1 complex in the pulmonary vascular wall visualized using the proximity ligation assay (PLA) method. Images are representative of 6 pulmonary arteries/at n=4 rats/group. Gray images were taken using light microscopy (×20) to visualize the tissue structure. The square represents the area of magnification (×100). The white marker corresponds to 25 μm. FIG. 4C shows the amplified signal from the RAGE-IMPA1 complex was registered in the media of hypertrophied pulmonary arteries, as seen on enlarged images from weeks 1 (Wk1) and 5 (Wk5). The dotted line is traced following the external lamina of the vessel. The arrowheads point to the endothelial cells with no PLA signal; white arrows point to the cells in the pulmonary artery media and adventitia cells with a bright PLA signal. IB, immunoblot.

FIGS. 5A, 5B, and 5C show pulmonary arterial hypertension (PAH) induces inositol monophosphatase 1 (IMPA1) translocation on a plasma membrane. FIG. 5A shows a validation method of separating the plasma membrane fraction and cytosolic fraction; samples were probed with plasma membrane protein sodium-potassium adenosine triphosphatase (Na,K ATPase). The 3 different presented sets of pulmonary lysates are collected from different animals, one set is shown in FIG. 5A. All sets show a strong signal from Na,K ATPase in the membrane but not a cytosolic fraction. Both receptors for advanced glycation endproducts (RAGE; FIG. 5B) and IMPA1 (FIG. 5C) accumulate in the membrane fraction isolated from pulmonary tissue of PAH rats. No significant changes were found in the levels of these proteins measured in the cytosolic fraction. The protein loading was normalized per total sample protein using stain-free imaging technology; n=6 for RAGE in the cytosolic fraction (FIG. 5B), and n=5 for all the rest groups. Results are expressed as box and whisker plots (boxes: 25th to 75th percentile of the data; whiskers: minimum to maximum; the line represents the median value). *P<0.05 vs. control group. Statistical analysis was performed using Bonferroni's multiple comparisons test for selected columns

FIGS. 6A, 6B, 6C, 6D, and 6E show the molecular modeling of receptors for advanced glycation endproducts (RAGE)-inositol monophosphatase 1 (IMPA1) interaction. FIG. 6A shows the docking of RAGE-originated peptide 365-RRQRRGEERKAP-376 (SEQ ID NO: 40) to IMPA1 structure revealed interaction with negatively charged COOH terminus of IMPA1. RAGE peptide binds into the cavity in IMPA1. Arginine (Arg) residue from RAGE peptide interacts with magnesium (Mg) ion at the active site. FIG. 6B shows the RAGE peptide binds to IMPA1 from the side that is opposite of the entrance into an active site. FIG. 6C shows an analysis of the surface electrostatic potential of IMPA1, showing the negatively charged region that binds RAGE peptide and the positively charged surface that can bind to the membrane. FIG. 6D illustrates the IMPA1 binding to the membrane based on the electrostatic map. FIG. 6E shows a schematic mechanism of RAGE-IMPA1 interaction. The negatively charged IMPA1 pocket interacts with a positively charged loop of the intracellular domain of RAGE. In this configuration, the positively charged surface of IMPA1 maintains IMPA1 attachment to the inner side of the plasma membrane.

FIGS. 7A, 7B, and 7C show an increased glucose uptake in the lungs starting from the early stage of pulmonary arterial hypertension (PAH). A time course of membrane translocation of 2 major glucose transporters, glucose transporter type 4 (GLUT4; FIG. 7A) and glucose transporter type 1 (GLUT1; FIG. 7B), revealed an early (week 1) significant accumulation of GLUT4 in the membrane fraction that was maintained throughout the study and accompanied by only mild changes in the cytosol. GLUT1 levels were progressively increased during the study and became significant by week 5 (Wk5) in both the membrane and cytosolic fractions. Protein loading was normalized per total sample proteins using stain-free imaging technology; n=5 rats in each group. FIG. 7C shows increased pulmonary levels of glucose 6-phosphate (G6P), confirming an upregulated lung glucose uptake starting from week 1 and providing the background for inositol monophosphatase 1 (IMPA1) activation; n=4 for control; n=8 for weeks 1 (Wk1) and 2 (Wk2); n=6 for the Wk5 group. Results are expressed as box and whisker plots (boxes: 25th to 75th percentile of the data; whiskers: minimum to maximum; the line represents the median value). *P<0.05 vs. control group. Statistical analysis was performed using Bonferroni's multiple-comparisons test for selected columns (FIG. 7A and 7B) and by Newman-Keuls multiple-comparisons test (FIG. 7C)

FIGS. 8A, 8B, 8C, 8D, and 8E shows upregulated inositol pathway and protein kinase B (Akt) activity in pulmonary arterial hypertension (PAH) rats. Activated in response to elevated glucose 6-phosphate (G6P) levels, inositol monophosphatase 1 (IMPA1) could stimulate inositol synthesis on a plasma membrane. FIG. 8A shows that indeed, the pulmonary phosphatidylinositol (3,4,5)-trisphosphate (PIP₃) was found to be strongly increased similar to G6P levels (FIG. 6C); n=6 rats in each group. The PAH has also induced an accumulation of catalytic subunits of phosphatidylinositol-3-kinase (PI3K) in the membrane fraction. However, phosphatidylinositol-4,5-bisphosphate 3-kinase catalytic subunit-γ (p100γ) was elevated at early and late stages (FIG. 8B), whereas phosphatidylinositol-4,5-bisphosphate 3-kinase catalytic subunit-α (p100α) levels increased only by week 5 (FIG. 8C); n=4 rats in each group. The binding of PIP₃ to the PH domain of Akt is known to induce Akt translocation on a plasma membrane and phosphorylation. FIG. 8D confirms that the increased formation of PIP₃ correlated with the depicted strong accumulation of Akt in the membrane fraction starting from the early stage of PAH, whereas the cytosolic levels of Akt were not affected. FIG. 8E shows the accumulation of Akt on the plasma membrane corresponded with the increased phosphorylation of Akt in the membrane but not a cytosolic fraction; n=4 rats in each group. Protein loading was normalized per total sample protein using stain-free imaging technology. Results are expressed as box and whisker plots (boxes: 25th to 75th percentile of the data; whiskers: minimum to maximum; the line represents the median value). *P<0.05 vs. control group. Statistical analysis was performed by Newman-Keuls multiple-comparisons test or using Bonferroni's Multiple comparisons test for selected columns (for control vs. week 1 in FIG. 8B and 8E). pS473Akt, protein kinase B phosphorylated at Ser473.

FIGS. 9A, 9B, and 9C show apoptosis-induced formation of a receptor for advanced glycation endproduct (RAGE)-inositol monophosphatase1 (IMPA1) complexes and protein kinase B (Akt) activation in vitro. FIG. 9A shows apoptosis-induced pulmonary artery vascular cells were used to prepare conditioned media. Cell media collected from apoptotic but not untreated control cells induced interaction between RAGE and IMPA1 (FIG. 9B) and activation of Akt (FIG. 9C) in naïve human pulmonary artery smooth muscle cells (HPASMC). Akt phosphorylation was significantly attenuated in the presence of selective IMPA1 inhibitor (L-690,330; 500 μM) and RAGE antagonist (RAP; 50 μM), confirming an important role of both proteins inactivation of Akt signaling; n=6/group (FIG. 9C) and n=5/group (FIG. 9B). Protein Loading was normalized per total sample protein using stain-free imaging technology. Results are expressed as box and whisker plots (boxes: 25th to 75th percentile of the data; whiskers: minimum to maximum; the line represents the median value). Statistical analysis was performed using an unpaired t-test (FIG. 9B) or Newman-Keuls Multiple-comparisons test (FIG. 9C). Apo, media was collected from apoptotic cells. *P<0.05 vs. control group; #P<0.05 vs. apoptosis group.

FIG. 10 shows a schematic representation of the proposed protein kinase B (Akt) activation in response to a receptor for advanced glycation endproducts (RAGE)-inositol monophosphatase 1 (IMPA1) interaction. Accumulation of glucose transporter type 4 (GLUT4) on a plasma membrane at the early stage of pulmonary arterial hypertension (PAH) increases glucose uptake and formation of glucose 6-phosphate (G6P). The increased levels of G6P stimulate IMPA1 activation and membrane translocation. Simultaneous activation of RAGE occurs in response to its interaction with damage-associated molecular patterns (DAMPs) that are released from dying cells. The formation of the RAGE-IMPA1 complex on a plasma membrane accelerates inositol synthesis and recycling. Phosphatidylinositol-3-kinase (PI3K) converts phosphatidylinositol 4,5-bisphosphate (PIP₂) into phosphatidylinositol (3,4,5)-trisphosphate (PIP₃), which mediates Akt membrane translocation and activation. The active Akt not only stimulates the activation of proliferative pathways but also ensures GLUT4 translocation on a plasma membrane, thus maintaining the feedforward stimulation of proliferative mechanisms. Thus, the co-occurrence of the early vascular damage and the glycolytic shift could be responsible for the persistent activation of uncontrolled growth in pulmonary vascular cells. Ins, inositol; IP, inositol monophosphate; IP, inositol 1,4,5-trisphosphate; p85, regulatory subunit of phosphatidylinositol-4,5-bisphosphate 3-kinase; p100, catalytic subunit of PIP3K; pS473, phosphorylated Ser473 of Akt.

FIGS. 11A, 11B, 11C, 11D, 11E, 11F, and 11G show the Anti-RAGE/IMPA1 peptide was designed based on the sequence of cytosolic tails of RAGE predicted to interact with IMPA1. FIG. 11A shows that by binding to IMPA1, the peptide blocks IMPA1 interaction with a full-size RAGE. FIG. 11B, 11C, and 11D show an injection of peptide (ILWQRRQRRG (SEQ ID NO: 1)) for 1 wk (5 μg/kg i.p. daily) to SU/Hx rats inhibits RAGE/IMPA1 co-IP (FIG. 11B) and reduces RVSP (FIG. 11C) and RV hypertrophy (FIG. 11D). N=2−4. *−p<0.05. FIG. 11E and 11F show that the anti-RAGE/IMPA1 peptide (3.3 mg/ml) reverses the highly proliferative phenotype of PAEC isolated from IPAH patients (FIG. 11E) but does not affect the growth of PAEC isolated from Control subjects (FIG. 11F). N=3. FIG. 11G shows that the anti-RAGE/IMPA1 peptide reverses the proliferative phenotype of HeLa cells. Thus, by blocking IMPA1/RAGE interaction, the inhibitory peptide prevents pathological cell growth without affecting normal cell proliferation

FIGS. 12A, 12B, 12C, 12D, 12E, 12F, 12G, 12H, 121, 12J, 12K, 12L, 12M, 12N, 120, 12P, 12Q, 12R, 12S, 12T, 12U, 12V, 12W, 12X, 12Y, 12Z, 12AA, 12AB, 12AC, and 12AD shows IC₅₀ concentration results of Cancer Cell Panel Screening Results.

FIG. 13 shows organ distribution among the most sensitive cell lines. The most sensitive cell line being SK-N-MC (Brain)—Neuroblastoma Cell Line second: NALM-6 (Blood)—Leukemia Cell Line, and third: LOU-NH91 (Lung)—Lung squamous cell carcinoma.

### DETAILED DESCRIPTION OF THE INVENTION

For purposes of summarizing the disclosure, certain aspects, advantages, and novel features of the disclosure are described herein. It is to be understood that not necessarily all such advantages may be achieved in accordance with any particular embodiments of the disclosure. Thus, the disclosure may be embodied or carried out in a manner that achieves or optimizes one advantage or group of advantages as taught herein without necessarily achieving other advantages as may be taught or suggested herein.

Additionally, although embodiments of the disclosure have been described in detail, certain variations and modifications will be apparent to those skilled in the art, including embodiments that do not provide all the features and benefits described herein. It will be understood by those skilled in the art that the present disclosure extends beyond the specifically disclosed embodiments to other alternative or additional embodiments and/or uses and obvious modifications and equivalents thereof. Moreover, while a number of variations have been shown and described in varying detail, other modifications, which are within the scope of the present disclosure, will be readily apparent to those of skill in the art based upon this disclosure. It is also contemplated that various combinations or sub-combinations of the specific features and aspects of the embodiments may be made and still fall within the scope of the present disclosure. Accordingly, it should be understood that various features and aspects of the disclosed embodiments can be combined with or substituted for one another in order to form varying modes of the present disclosure. Thus, it is intended that the scope of the present disclosure herein disclosed should not be limited by the particular disclosed embodiments described herein.

As used herein, the singular forms “a,” “an,” and “the” are intended to include the plural forms as well, unless the context clearly indicates otherwise. Furthermore, to the extent that the terms “including,” “includes,” “having,” “has,” “with,” or variants thereof are used in either the detailed description and/or the claims, such terms are intended to be inclusive in a manner similar to the term “comprising.”

As used herein, the terms “subject” and “patient” are used interchangeably. As used herein, a subject can be an animal (amphibian, reptile, avian, fish, or mammal) such as a non-primate (e.g., cows, pigs, horses, cats, dogs, rats, etc.) or a primate (e.g., monkey, ape, and human). In specific embodiments, the subject is a human. In one embodiment, the subject is a mammal (e.g., a human) having a disease, disorder, or condition described herein. In another embodiment, the subject is a mammal (e.g., a human) at risk of developing a disease, disorder, or condition described herein. In certain instances, the term patient refers to a human under medical care.

The terms “treating” or “treatment” refer to any indicia of success or amelioration of the progression, severity, and/or duration of a disease, pathology, or condition, including any objective or subjective parameter such as abatement; remission; diminishing of symptoms or making the injury, pathology or condition more tolerable to the patient; slowing in the rate of degeneration or decline; making the final point of degeneration less debilitating; or improving a patient's physical or mental well-being.

The terms “manage,” “managing,” and “management” refer to preventing or slowing the progression, spread, or worsening of a disease or disorder or of one or more symptoms thereof. In certain cases, the beneficial effects that a subject derives from a prophylactic or therapeutic agent do not result in a cure of the disease or disorder.

The term “effective amount” as used herein refers to the amount of a therapy or medication which is sufficient to reduce and/or ameliorate the severity and/or duration of a given disease, disorder, or condition and/or a symptom related thereto. This term also encompasses an amount necessary for the reduction or amelioration of the advancement or progression of a given disease (e.g., cancer), disorder or condition, reduction or amelioration of the recurrence, development or onset of a given disease, disorder or condition, and/or to improve or enhance the prophylactic or therapeutic effect(s) of another therapy. In some embodiments, “effective amount” as used herein also refers to the amount of therapy provided herein to achieve a specified result.

As used herein, and unless otherwise specified, the term “therapeutically effective amount” is an amount sufficient enough to provide a therapeutic benefit in the treatment or management of a disease or to delay or minimize one or more symptoms associated with the presence of the cardiovascular disease. A therapeutically effective amount of an agent means an amount of therapeutic agent, alone or in combination with other therapies, which provides a therapeutic benefit in the treatment or management of the disease. The term “therapeutically effective amount” can encompass an amount that improves overall therapy, reduces or avoids symptoms or causes of cardiovascular disease, or enhances the therapeutic efficacy of another therapeutic agent.

The terms “administering”, and “administration” refer to methods of providing a pharmaceutical preparation to a subject. Such methods are well known to those skilled in the art and include, but are not limited to, administering the compositions orally, intranasally, parenterally (e.g., intravenously and subcutaneously), by intramuscular injection, by intraperitoneal injection, intrathecally, transdermally, extracorporeally, topically or the like.

Pulmonary arterial hypertension (PAH) is a lethal disease characterized by progressive pulmonary vascular remodeling. The receptor for advanced glycation endproducts (RAGE) plays an essential role in PAH by promoting the proliferation of pulmonary vascular cells. RAGE is also known to mediate the activation of Akt Signaling, although the particular molecular mechanism remains unknown. The progressive angioproliferative PAH was induced in 24 female Sprague-Dawley rats (n=8/group) that were randomly assigned to develop PAH for 1, 2, or 5 wk [right ventricle systolic pressure (RVSP) 56.5±3.2, 63.6±1.6, and 111.1±4.5 mmHg, respectively, vs. 22.9±1.1 mmHg in controls]. PAH triggered early and late episodes of apoptosis in rat lungs accompanied by RAGE activation. Mass Spectrometry analysis has identified IMPA1 as a novel PAH-specific interacting partner of RAGE. The Proximity ligation assay (PLA) confirmed RAGE/IMPA1 complex formation in the pulmonary artery wall. Activation of IMPA1 in response to increased glucose 6-phosphate (G6P) is known to play a critical role in inositol synthesis and recycling. Indeed, a threefold increase in G6P (P=0.0005) levels in lungs of PAH rats starting from week 1 that correlated with accumulation of phosphatidylinositol (3,4,5)-trisphosphate (PIP3), membrane translocation of PI3K, and a threefold increase in membrane Akt levels (P=0.02) and Akt phosphorylation.

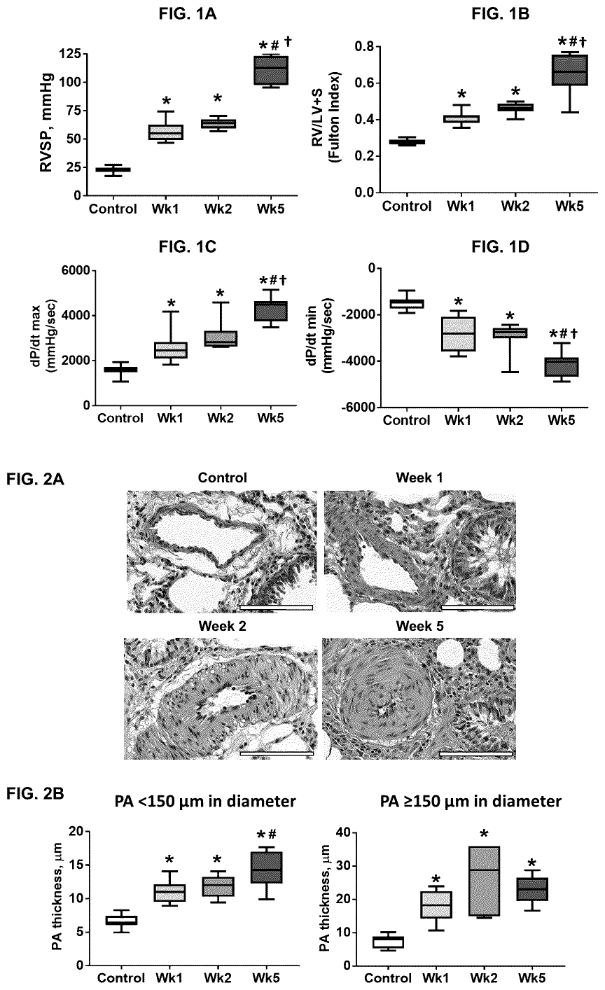

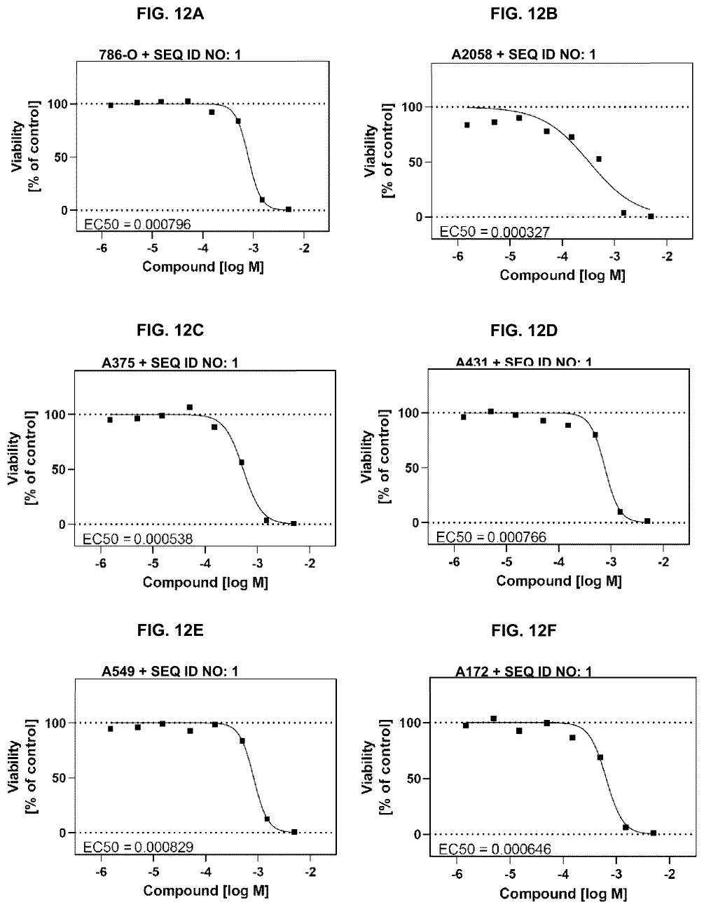

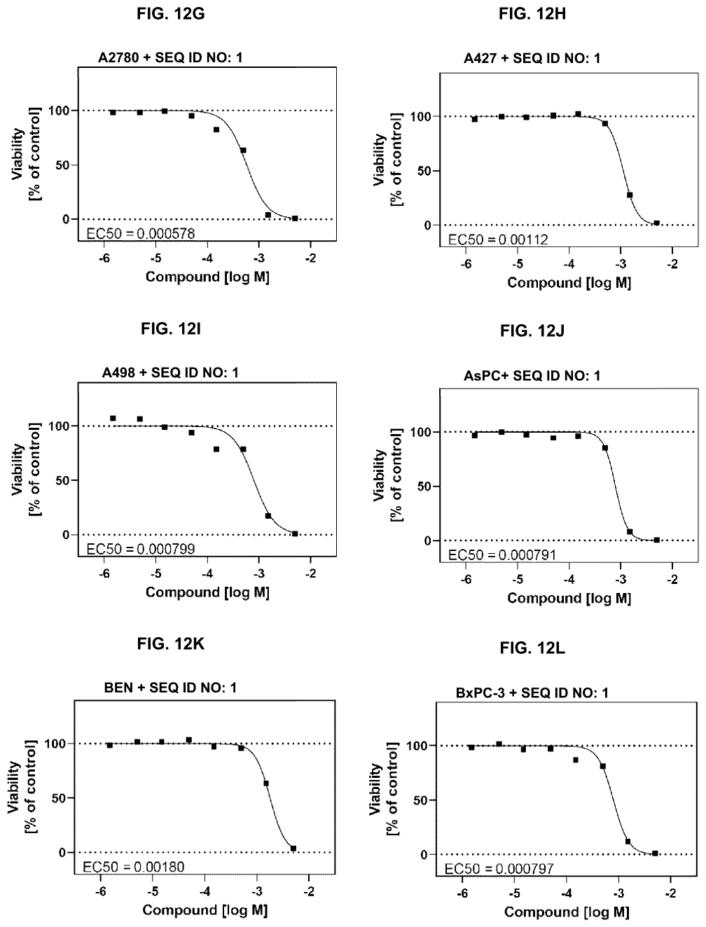

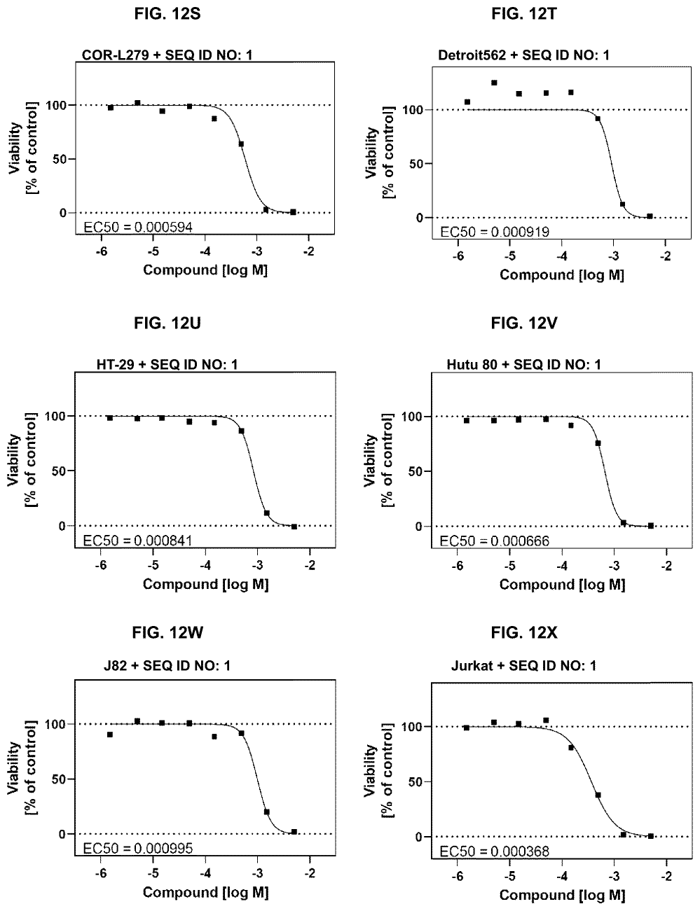

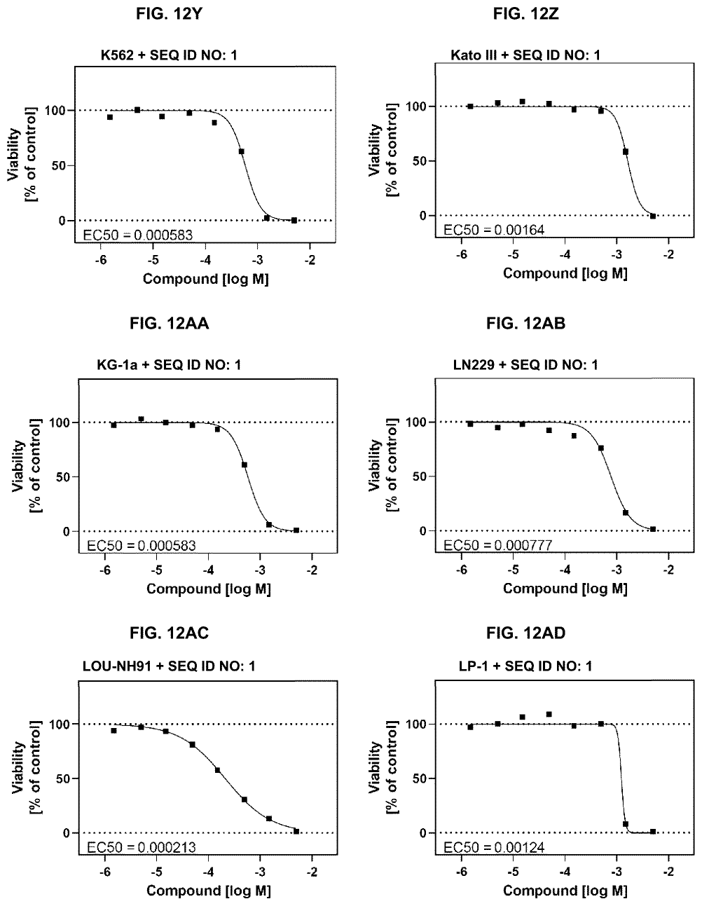

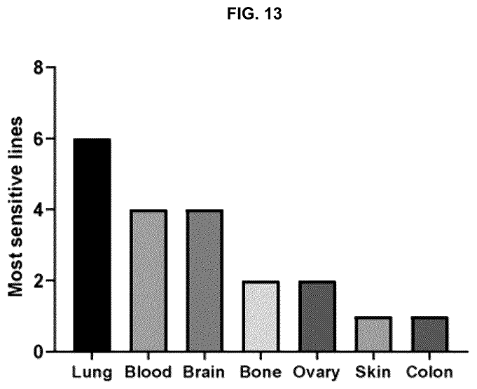

Referring now to FIGS. 1A-13, the present invention features a peptide comprising a sequence at least 80% identical to ILWQRRQRRG (SEQ ID NO: 1). In other embodiments, the present invention features a peptide comprising a sequence at least 80% identical to ILWQRRQRRGEERKAP (SEQ ID NO: 2).

The present invention features an antiproliferative peptide comprising a sequence at least 80% identical to ILWQRRQRRG (SEQ ID NO: 1) or ILWQRRQRRGEERKAP (SEQ ID NO: 2). In some embodiments, the peptide inhibits interaction between inositol monophosphatase 1 (IMPA1) and receptor for advanced glycation endproducts (RAGE).

The present invention features an antiproliferative peptide comprising a sequence at least 80% identical to ILWQRRQRRG (SEQ ID NO: 1) or ILWQRRQRRGEERKAP (SEQ ID NO: 2) or a variant thereof. In some embodiments, the peptide inhibits interaction between inositol monophosphatase 1 (IMPA1) and receptor for advanced glycation endproducts (RAGE).

In some embodiments, the peptides described herein comprise a sequence at least 50% identical to ILWQRRQRRG (SEQ ID NO: 1). In some embodiments, the peptides described herein comprise a sequence at least 60% identical to ILWQRRQRRG (SEQ ID NO: 1). In some embodiments, the peptides described herein comprise a sequence at least 70% identical to ILWQRRQRRG (SEQ ID NO: 1). In some embodiments, the peptides described herein comprise a sequence at least 80% identical to ILWQRRQRRG (SEQ ID NO: 1). In some embodiments, the peptides described herein comprise a sequence at least 90% identical to ILWQRRQRRG (SEQ ID NO: 1). In some embodiments, the peptides described herein comprise a sequence at least 100% identical to ILWQRRQRRG (SEQ ID NO: 1).

In some embodiments, the peptides described herein comprise a sequence at least 50% identical to ILWQRRQRRGEERKAP (SEQ ID NO: 2). In some embodiments, the peptides described herein comprise a sequence at least 56% identical to ILWQRRQRRGEERKAP (SEQ ID NO: 2). In some embodiments, the peptides described herein comprise a sequence at least 62% identical to ILWQRRQRRGEERKAP (SEQ ID NO: 2). In some embodiments, the peptides described herein comprise a sequence at least 68% identical to ILWQRRQRRGEERKAP (SEQ ID NO: 2). In some embodiments, the peptides described herein comprise a sequence at least 75% identical to ILWQRRQRRGEERKAP (SEQ ID NO: 2). In some embodiments, the peptides described herein comprise a sequence at least 81% identical to ILWQRRQRRGEERKAP (SEQ ID NO: 2). In some embodiments, the peptides described herein comprise a sequence at least 87% identical to ILWQRRQRRGEERKAP (SEQ ID NO: 2). In some embodiments, the peptides described herein comprise a sequence at least 93% identical to ILWQRRQRRGEERKAP (SEQ ID NO: 2). In some embodiments, the peptides described herein comprise a sequence at least 100% identical to ILWQRRQRRGEERKAP (SEQ ID NO: 2).

In some embodiments, the peptides described herein comprise at least one modification. In other embodiments, the peptides described herein comprise at least two modifications. In further embodiments, the peptides described herein comprise at least three modifications. In some embodiments, the peptides described herein comprise at least four modifications. In some embodiments, the modification is a substitution. In some embodiments, the modification is a substitution or a deletion.

In some embodiments, the I amino acid is substituted with a V or an L amino acid. In some embodiments, the L amino acid is substituted with an I or a V amino acid. In some embodiments, the W amino acid is substituted with an F or a Y amino acid. In some embodiments, at least one of the Q amino acids is substituted with an N amino acid. In some embodiments, at least one of the R amino acids is substituted with a K amino acid. In some embodiments, the G amino acid is substituted with an A amino acid. In some embodiments, at least one of the E amino acids is substituted with a D amino acid. In some embodiments, the K amino acid is substituted with an R amino acid. In some embodiments, the A amino acid is substituted with a G amino acid. In some embodiments, the P amino acid is substituted with an H amino acid.

TABLE 1
Non-limiting examples of a peptide comprising a singular substitution
modification:

| SEQ ID NO: | Sequence | SEQ ID NO: | Sequence |
|---|---|---|---|
| 3 | LLWQRRQRRGEERKAP | 21 | ILWQRRQKRGEERKAP |
| 4 | VLWQRRQRRGEERKAP | 22 | ILWQRRQRKGEERKAP |
| 5 | LLWQRRQRRG | 23 | ILWQRRQRRGEEKKAP |
| 6 | VLWQRRQRRG | 24 | ILWQKRQRRG |
| 7 | IIWQRRQRRGEERKAP | 25 | ILWQRKQRRG |
| 8 | IVWQRRQRRGEERKAP | 26 | ILWQRRQKRG |
| 9 | IIWQRRQRRG | 27 | ILWQRRQRKG |
| 10 | IVWQRRQRRG | 28 | ILWQRRQRRAEERKAP |
| 11 | ILFQRRQRRGEERKAP | 29 | ILWQRRQRRA |
| 12 | ILYQRRQRRGEERKAP | 30 | ILWQRRQRRGDERKAP |
| 13 | ILFQRRQRRG | 31 | ILWQRRQRRGEDRKAP |
| 14 | ILYQRRQRRG | 32 | ILWQRRQRRGEERRAP |
| 15 | ILWNRRQRRGEERKAP | 33 | ILWQRRQRRGEERKGP |
| 16 | ILWQRRNRRGEERKAP | 34 | ILWQRRQRRGEERKAH |
| 17 | ILWNRRQRRG | 35 | RRQRRGEERKAPGP |
| 18 | ILWQRRNRRG | 36 | ILWQRRQRRG |
| 19 | ILWQKRQRRGEERKAP | 37 | PGPILWQRRQRRG |
| 20 | ILWQRKQRRGEERKAP | 38 | LLWQRRQRRG |

*Note:
Bolded amino acids indicate those that have been modified to D-amino acids.

In some embodiments, the peptides described herein are selected from a group consisting of SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, and SEQ ID NO: 38.

In some embodiments, the peptides described herein comprise a sequence at least 50% identical to the aforementioned sequences. In some embodiments, the peptides described herein comprise a sequence at least 56% identical to the aforementioned sequences. In some embodiments, the peptides described herein comprise a sequence at least 60% identical to the aforementioned sequences. In some embodiments, the peptides described herein comprise a sequence at least 62% identical to the aforementioned sequences. In some embodiments, the peptides described herein comprise a sequence at least 68% identical to the aforementioned sequences. In some embodiments, the peptides described herein comprise a sequence at least 70% identical to the aforementioned sequences. In some embodiments, the peptides described herein comprise a sequence at least 75% identical to the aforementioned sequences. In some embodiments, the peptides described herein comprise a sequence at least 80% identical to the aforementioned sequences. In some embodiments, the peptides described herein comprise a sequence at least 81% identical to the aforementioned sequences. In some embodiments, the peptides described herein comprise a sequence at least 87% identical to the aforementioned sequences. In some embodiments, the peptides described herein comprise a sequence at least 90% identical to the aforementioned sequences. In some embodiments, the peptides described herein comprise a sequence at least 93% identical to the aforementioned sequences. In some embodiments, the peptides described herein comprise a sequence at least 100% identical to the aforementioned sequences.

In some embodiments, the peptides described herein further comprise a cell membrane crossing sequence. In some embodiments, the cell membrane crossing sequence comprises GRKKRRQRRRPQ (SEQ ID NO: 39). In some embodiments, the cell membrane crossing sequence comprises a sequence at least 92% identical to SEQ ID NO: 39. In some embodiments, the cell membrane crossing sequence comprises a sequence at least 83% identical to SEQ ID NO: 39. In some embodiments, the cell membrane crossing sequence comprises a sequence at least 75% identical to SEQ ID NO: 39. In some embodiments, the cell membrane crossing sequence comprises a sequence at least 67% identical to SEQ ID NO: 39. Other cell membrane crossing sequences (i.e., cell membrane penetrating sequence) may be used in accordance with the present invention.

In some embodiments, the cell membrane crossing sequence is modified. In some embodiments, the cell membrane crossing sequence is modified with a chemical group. In some embodiments, the chemical group is an amidation. In some embodiments, the cell membrane crossing sequence comprises modified amino acids (e.g., phosphorylated). In some embodiments, the chemical group is a triphenylphosphonium group or derivative thereof. In some embodiments, the membrane crossing sequence is shortened. For example, the membrane crossing sequence is shortened by one amino acid, two amino acids, or three amino acids. In some embodiments, the membrane crossing sequence (e.g., SEQ ID NO: 39) is shortened by two or three positively charged amino acids (e.g., R, K, H). In other embodiments, the membrane crossing sequence comprises extra amino acid residues. In some embodiments, the membrane crossing sequence comprises one extra amino acid residue. In some embodiments, the membrane crossing sequence comprises two extra amino acid residues. In some embodiments, the membrane crossing sequence comprises three extra amino acid residues. In some embodiments, the membrane crossing sequence comprises five extra amino acid residues. The crossing membrane sequence may comprise more than five, ten, or twenty extra amino acid residues.

In some embodiments, the amino acids are D-amino acids, L-amino acids, or combinations thereof. In some embodiments, the amino acids are unnatural amino acids, modified amino acids (e.g., phosphorylated), or a combination thereof. In some embodiments, the N-terminal or the C-terminal of a peptide described herein is modified. In some embodiments, the modification comprises adding a chemical moiety, a membrane crossing sequence, a chemical group to the N-terminal or the C-terminal. Non-limiting examples of modifications include but are not limited to a triphenylphosphonium group, a TAT sequence, polyimide or polyethylene glycol, or other hydrophobic and positively charged chemical groups. In some embodiments, the modification is a TAT sequence or a triphenylphosphonium group.

The present invention may feature a method of treating a disease with an increase in proliferation (e.g., proliferative disorders) in a subject in need thereof, the method comprising administering a therapeutically effective amount of a peptide as described herein.

The present invention may also feature a method of treating a proliferation disease in a subject in need thereof, the method comprising administering a therapeutically effective amount of a peptide as described herein.

In some embodiments, the disease is pulmonary arterial hypertension (PAH). In other embodiments, the disease is cancer, e.g., bladder cancer, breast cancer, lung cancer, prostate cancer, cervical cancer, colorectal cancer, kidney cancer, melanoma, non-Hodgkin lymphoma, leukemia, endometrial cancer, pancreatic cancer, thyroid cancer, liver cancer, etc. In some embodiments, the disease is a proliferative disorder, e.g., diabetic retinopathy.

The present invention may further feature a method of inhibiting an interaction between inositol monophosphatase 1 (IMPA1) and receptor for advanced glycation endproducts (RAGE) in vitro, the method comprising administering a peptide as described herein to a cell or an in vitro system.

The present invention features a method of inhibiting an interaction between inositol monophosphatase 1 (IMPA1) and receptor for advanced glycation endproducts (RAGE) in a subject, the method comprising administering a peptide as described herein to the subject.

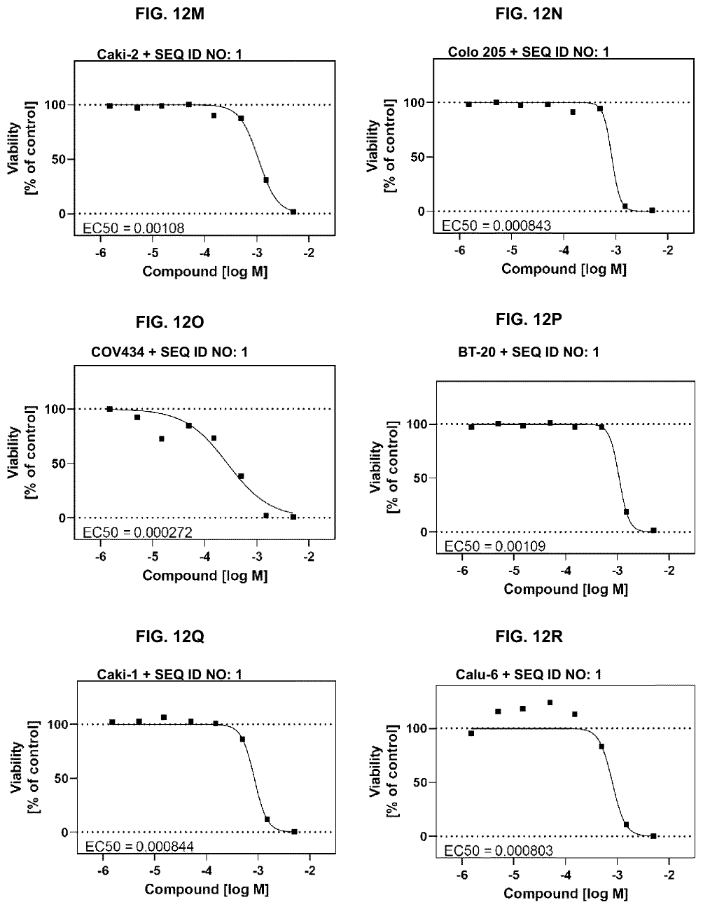

Referring to FIG. 12A-12AD, the present invention may also feature an antiproliferative peptide comprising a sequence at least 80% identical to ILWQRRQRRG (SEQ ID NO: 1) or ILWQRRORRGEERKAP (SEQ ID NO: 2) for use in a method for treatment of proliferative disease. In some embodiments, the disease is pulmonary arterial hypertension (PAH). In some embodiments, the disease is cancer, e.g., bladder cancer, breast cancer, lung cancer, prostate cancer, cervical cancer, colorectal cancer, kidney cancer, melanoma, non-Hodgkin lymphoma, leukemia, endometrial cancer, pancreatic cancer, thyroid cancer, liver cancer, etc.

The present invention may feature an antiproliferative peptide comprising a sequence at least 80% identical to ILWQRRQRRG (SEQ ID NO: 1) or ILWQRRQRRGEERKAP (SEQ ID NO: 2) for use in a method for treatment of pulmonary arterial hypertension (PAH).

### EXAMPLE

The following is a non-limiting example of the present invention. It is to be understood that said example is not intended to limit the present invention in any way. Equivalents or substitutes are within the scope of the present invention.

Rat models of pulmonary hypertension: Thirty-two Sprague-Dawley female rats (200-250 g) were purchased from Charles River (Wilmington, MA). Animals were kept in a 12-h:12-h light-dark cycle and received standard rodent food and water ad libitum. All experimental procedures were approved by the University of Arizona's Institutional Animal Care and Use Committee. To induce PAH, rats received either the vehicle or a single dose of the VEGF receptor 2 antagonist SU5416, 50 mg/kg sc, as previously described. On the day of injection, the PAH rats were placed in a hypoxic chamber (BioSpherix, Redfield, NJ), and the oxygen was maintained at the level of 10±0.5%. The O₂ and CO₂ concentrations were continuously monitored (PROOX 110 BioSpherix oxygen controller and LB-2 CO₂ analyzer; Sensormedics).

The animals were analyzed after 1 or 2 weeks of exposure to hypoxia or after 5 weeks (i.e., 3 weeks of hypoxia and 2 weeks of normoxia). The control rats were kept under normoxic conditions for the duration of the study (5 weeks). At the end of each study period, animals were anesthetized (in actin, 100 mg/kg ip) and instrumented for measurement of right ventricle (RV) hemodynamics. Briefly, a PE-240 polyethylene tube was inserted into the trachea to facilitate breathing. A customized pressure transducer catheter (SPR-513; Millar Instruments, Houston, TX), connected to a Millar Transducer Control Unit TC-510 and PL3504 PowerLab 4/35 data acquisition system (ADInstruments, Colorado Springs, CO) was inserted into RV via the right jugular vein and right atrium. A 30-minutes stabilization period was permitted before a 30-minute recording of RV pressure. At the end of pressure recording, the trachea catheter was connected to a Harvard Rodent Ventilator (Model 683; Harvard Apparatus, South Natick, MA), the thorax was opened, the left atrium was cut, and the lungs were flushed with saline (0.9% sodium chloride) via a needle inserted into RV. Animals were euthanized by removing heart/lung block; lungs, RV, and left ventricle plus septum (LV+S) were dissected and weighed. For histological examination, the left lung was fixed in formalin and embedded in paraffin. For biochemical analysis, the right lung was quick-frozen and stored at −80° C. for biochemical analysis.

Histological analysis: For the morphometric assessment of pulmonary vessels, 5 μm tissue sections were dewaxed and stained with hematoxylin and eosin by HistoWiz using standard operating procedures and a fully automated workflow. Twenty transversely sectioned pulmonary arteries per animal (from 6 to 8 rats/group) were randomly selected from the whole-slide ×40 digitized image created by HistoWiz using Aperio AT2 scanner (Leica Biosystems). The morphometric analysis was done by an investigator blinded for the animal group, as previously described. The pulmonary artery wall thickness was measured perpendicular to the circumference of the vessel from the endothelium to the outer edge of the smooth muscle layer using ImageJ software. The average of four measurements at a different location of the circumference of each pulmonary artery was quantified. The pulmonary arteries were divided into two groups, small (an external diameter of <150 μm) and larger (an external diameter of ≥150 μm) vessels, and the data were presented separately for each group.

In situ apoptosis detection: Apoptosis of pulmonary arteries was visualized using Click-iT Plus terminal deoxynucleotidyl transferase-dUTP nick end labeling (TUNEL) assay (ThermoFisher Scientific) according to the manufacturer's protocol, with some modifications. Briefly, sections were deparaffinized in two changes of xylene for 5 min each, hydrated with two changes of 100, 95, 70, and 50% ethanol, 0.85% NaCl for 5 minutes, and washed in PBS for 5 minutes. Tissue sections were fixed with 4% paraformaldehyde for 15 minutes at room temperature and treated with proteinase K-ready solution for 2 minutes. Fixative solution (e.g., 4% paraformaldehyde) was applied for 5 minutes, followed by incubation in 100 μl of TdT reaction buffer for 10 minutes at 37° C. and Td reaction mixture for 15 minutes at 37° C. After washing, the slides were incubated in Click-iT TUNEL Colorimetric Reaction cocktail for 30 minutes at 37° C., washed again, and incubated in 100 μl of Streptavidin-Peroxidase Conjugate at room temperature for 30 minutes in a humidified chamber in the dark. After the next serial washes, the slides were covered with 100 μl of the DAB Reaction Mixture, washed, counterstained with hematoxylin for 5 seconds, rinsed, and dehydrated. Coverslips were applied with the xylene-based mounting medium.

Duolink in situ proximity ligation assay: The RAGE/IMPA1 complex formation was visualized using proximity ligation assay according to a standard protocol (Sigma-Aldrich, St. Louis, MO). Briefly, slides were deparaffinized, and antigen retrieval was performed in 10 mM sodium citrate, pH 6.0, at 125° C. for 5 minutes and incubated with anti-RAGE rabbit polyclonal (Abcam, Cambridge, MA) and anti-IMPA1 mouse monoclonal (Santa Cruz Biotechnology, Santa Cruz, CA) antibodies diluted 1:100. Incubation with Far-Red PLA probes, followed by ligation and amplification, was performed according to the manufacturer's manual. Slides were dried and mounted with Prolong Diamond antifading agent with DAPI (Invitrogen). Microscopy was performed using a Leica 6000 inverted microscope, Y5 (Cy5) filter cube for PLA probe, and D cube for DAPI at ×40 and ×100 magnification. This technology allows a pair of oligonucleotide-labeled antibodies (PLA probes) attached to the secondary antibody to generate an amplified signal only when the probes are in close proximity (<40 nm). Six pulmonary arteries per animal (from 4 lung preparations per experimental group) were randomly selected and imaged.

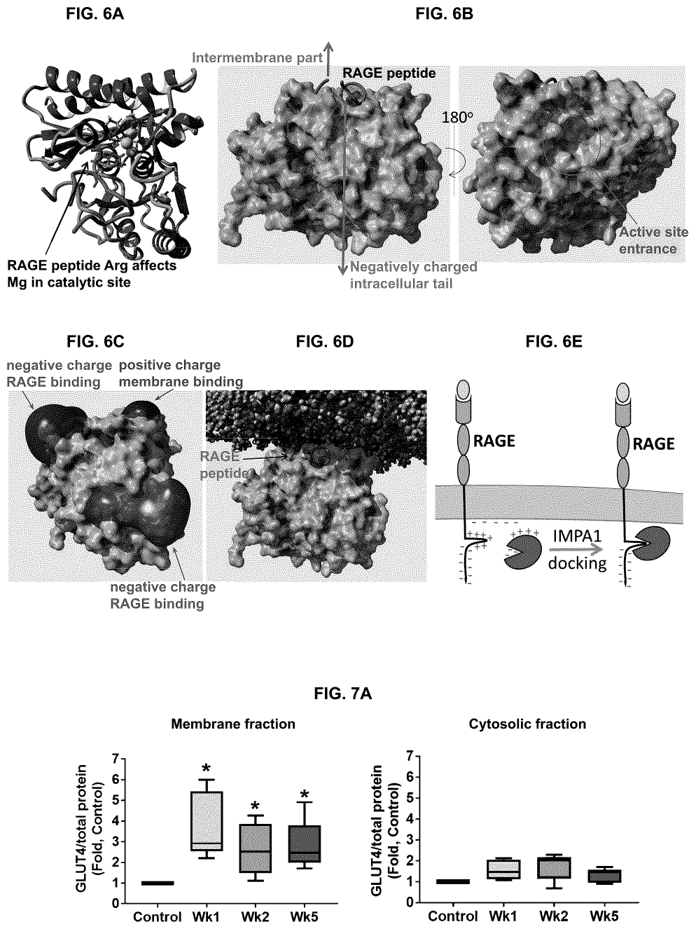

Western blot and immunoprecipitation: For an analysis of the total lung proteins, lung tissues were lysed. According to the manufacturer's protocol, the membrane proteins were isolated using Mem-PER Plus Membrane Protein Extraction Kit (ThermoFisher Scientific, Rockford, IL). Briefly, 20-40 mg of lung tissue was quickly washed in Cell WashSolution to remove any blood components, transferred to permeabilization buffer mixed with protease and phosphatase inhibitor cocktails, homogenized using Fisher Homogenizer 850 for 1 minute, and shaken in UltraCruz Shaker at 0° C. for 10 minutes. The homogenate was centrifuged at 16,000 g for 15 minutes to pellet permeabilized cells. The supernatant containing cytosolic proteins was carefully transferred to a separate tube. The pellet was suspended in a solubilization buffer with a protease and phosphatase inhibitor cocktail and shaken at 4° C. for 30 minutes. The lysate was centrifuged at 16,000 g for 15 minutes, and the supernatant containing solubilized membrane-associated proteins was transferred to separate tubes and used for protein analysis. Briefly, the samples were incubated with 6× Laemmli sample buffer (Boston Bioproducts, Ashland, MA), for 5 minutes at 95° C., loaded on the 4-20% Mini-PROTEAN TGXStain-Free gels (Bio-Rad Laboratories, Hercules, CA), and electrophoretically separated and transferred using PowerPac Universal power supply and Trans-Blot Turbo transferring system (Bio-Rad Laboratories). Membranes were probed using antibodies against cleaved caspase 3, Akt1, pS473-Akt, Na,K-ATPase (CellSignaling Technology, Danvers, MA), RAGE, GLUT1, GLUT4, p110γ, p110α (Santa Cruz Biotechnology, Santa Cruz, CA), MyD88, and IMPA1 (Abcam, Cambridge, MA). The reactive bands were visualized using a chemiluminescent protocol, recorded with the ChemiDoc MP Imaging System (Bio-RadLaboratories, Hercules, CA), and analyzed using Image Laboratory software. The protein loading was normalized per total sample protein using free stain gels. The efficiency of tissue fractionation was validated by using the antibodies against plasma membrane marker Na, K-ATPase (FIG. 5A). Some membranes were stripped and reprobed for more than one protein.

For immunoprecipitation (IP), RAGE antibodies conjugated to agarose beads were used according to the manufacturer's (Santa Cruz Biotechnology) protocol. Lung tissue lysate (300 μg of total protein) was rotated at room temperature with 20 μl of bead solution for 2 hours. Beads were collected by brief centrifugation (30 seconds at 1,000 g), washed four times with lysis buffer and PBS, resuspended with 30 μl of 2× Laemmli sample buffer, and boiled for 5 minutes at 95° C. The supernatant collected by centrifugation was used for Western Blot analysis as described.

Antibody validation: Antibodies used in this study have undergone previous validation.

Proteomic analysis of RAGE Co-IP: As described above, the lungs lysate from the control and week 5 groups were subjected to IP. Proteins were separated on 4-20% Mini-PROTEAN TGX Stain-Free gel and stained with Bio-Safe Coomassie G-250 Stain (Bio-Rad, Hercules, CA). For the proteome analysis, each gel lane was cut into three slices to target proteins in the 10- to 50-kDa range. Destaining, in-gel digestion, and peptide extraction steps were done as previously published. The dried peptides were resuspended in 6 μl of 0.1% formic acid, then sonication for 2 minutes. HPLC-ESI-MS/MS was performed in positive ion mode on a Thermo Scientific Orbitrap Fusion Lumos tribrid mass spectrometer fitted with an EASY-Spray Source (Thermo Scientific, San Jose, CA). NanoLC was performed using a Thermo Scientific UltiMate 3000 RSLCnano System with an EASY Spray C18 LC column (75 cm×75 μm inner diameter, packed with PepMap RSLC C18 material, 2 μm, cat. no. ES805; Thermo Scientific), loading phase for 15 minutes at 0.300 μl/min, mobile phase, and a linear gradient of 1-34% Solvent B in 119 minutes at 0.220 μl/min, followed by a step to 95% buffer B over 4 minutes at 0.220 μl/min, hold for 5 minutes at 0.250 μl/min, and then a step to 1% buffer B over 5 minutes at 0.250 μl/min, and a final hold for 10 minutes (total run 159 min): buffer A=100% H2O in 0.1% FA, buffer B=80% ACN in 0.1% FA. All solvents were liquid chromatography-mass spectrometry grade. Spectra was acquired using XCalibur version 2.3 (Thermo Scientific). A “top speed” data-dependent MS/MS analysis was performed. Dynamic exclusion was enabled with a repeat count of 1, a repeat duration of 30 seconds, and an exclusion duration of 60 seconds. Tandem Mass spectra were extracted from Xcalibur “RAW” files, and charge states were assigned using the ProteoWizard 3.0 msConvert script using the default parameters. The fragment mass spectra were then searched against the rat SwissProt_2016_10 database (7983 entries) using Mascot (version 2.6.0; MatrixScience, London, UK) using the default probability cutoff score. The search variables used were 10 ppm mass tolerance for precursor ion masses and 0.5 Da for product ion masses, digestion with trypsin, a maximum of two missed tryptic cleavages, and variable modifications of oxidation methionine and phosphorylation of serine, threonine, and tyrosine. Cross-correlation of Mascot search results with X! Probability assessments of peptide assignments and protein identifications were made using Scaffold. To enrich peptide coverage, a threshold was set to ≥0% probability, and the protein threshold was set to P>99%.

Homology modeling and docking: To identify the interaction sites between IMPA1 and RAGE, a homology model of IMPA1 was built. The available structure of IMPA1 (PDB ID: 2BJI) was used. To model the structure, the Yasara Structure software package was utilized. The geometry of the reconstructed region of human IMPA1 was automatically optimized using the steepest descent energy minimization algorithm in the solvent implicit model. In the refined structure of IMPA1, the electrostatic potential was analyzed on the surface and the RAGE peptide docking to IMPA1 using embedded in Yarasa algorithms.

Glucose 6-phosphate quantification: Glucose 6-phosphate (G6P) fluorometric assay from Abnova (Littleton, CO) was used to detect the levels of G6P according to the manufacturer's protocol. Briefly, 100 mg of lung tissue was rapidly homogenized in ice-cold assay buffer. 10K cutoff filters (Amicon) were used for deproteinization of the samples. After incubation with enzymes, fluorescence measurement using Ex/Em= 535/587 nm was done on the Biotek Synergy H1m plate reader.

Phosphatidylinositol 3, 4, 5-triphosphate quantification: Phosphatidylinositol 3,4,5-trisphosphate (PIP₃) assay kit (Echelon Biosciences) was used for the quantification of PIP₃ in tissue samples. Lipid fraction was extracted from 100 mg of lung tissue and subjected to manufacturer-supplied protocol. Absorbance at 450 nm was measured on the Biotek Synergy H1m multi-plate reader.

Cell culture: Human pulmonary artery endothelial cells (HPAEC; cat. no. 3100, lot no. 3904) and human pulmonary artery smooth muscle cells (HPASMC; cat. no. 3110, lot no. 0294) were purchased from ScienCell (Carlsbad, CA), one donor for each cell type. These primary vascular cells are nontransformed, nonimmortalized human cells isolated directly from lung tissue. HPAEC (passages 4-6) were cultured in ECM growth media (cat. no. 1001; ScienCell) supplemented with 5% FBS (cat. no. 25-514H; GeneseeScientific) and penicillin-streptomycin (cat. no. 15140-122; Gibco) in a humidified incubator (21% O, 5% CO) at 37° C. To induce apoptosis, the medium was changed to serum-free ECM for 48-72 hours. HPASMC were cultured in DMEM supplemented with 10% FBS and 4.5 g/L glucose. Apoptosis of HPASMC was induced by changing the media to DMEM with 5% FBS and 0.1 g/L glucose for 48-72 h. The level of apoptosis was quantified using Apoptosis and Necrosis Quantification Kit (Biotum, Fremont, CA) according to the manufacturer's protocol. Briefly, cells were collected with trypsin, washed with PBS, and resuspended in 50 μl of reaction mix containing a buffer, FITC-Annexin V, and Ethidium homodimer. After 25 minutes of incubation in the dark at room temperature, samples were diluted with 200 μl of the kit 1× buffer. The analysis was performed using NovoCyte Flow Cytometer (ACEA Biosciences, San Diego, CA). The conditioned medium was collected from apoptotic and untreated cells and centrifuged at 12,000 g for 10 minutes at 4° C. The medium collected from apoptotic HPASMC was supplemented back with glucose (4.5 g/L), and the medium collected from apoptotic HPAEC was supplemented with 2% FBS. The naïve HPASMC were treated with conditioned media or media premixed with selective IMPA1 inhibitor (cat. no.sc-202685AL-690,330, 500 μM; Santa Cruz Biotechnology) or RAGE antagonist peptide (RAP; 50 μM, EMD Millipore, Burlington, MA, cat no. 553031). After 24 h of incubation, the medium was removed, and cells were washed with PBS and frozen for the future Western blot (WB) analysis as described above. Alternatively, as described above, the naïve HPASMC treated by control and apoptotic conditioned media were used for RAGE immunoprecipitation.

Statistical analysis: Statistical calculations were performed using the GraphPad Prism software version 7.04. The mean value (±SE) was calculated for all samples, and significance was determined by either the unpaired t-test or analysis of variance (ANOVA). For ANOVA, Newman-Keuls or Bonferroni multiple comparison tests to compare the selected pairs of columns were used. A value of P<0.05 was considered significant. The Grubbs test (extreme studentized deviation) was used to determine the significant outliers. This criterion was predetermined before the initiation of the data analysis.

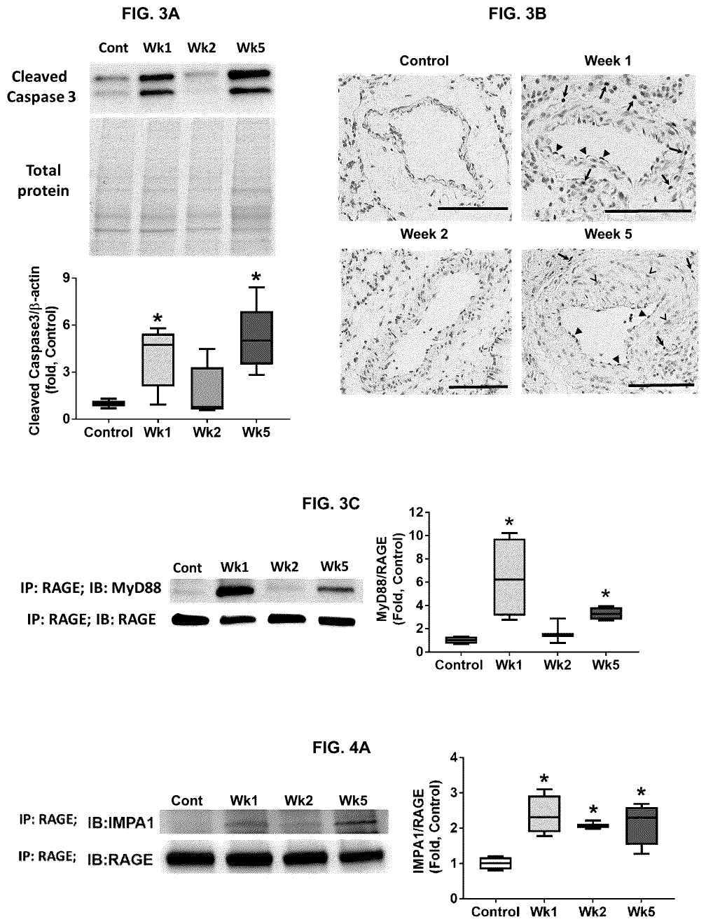

Time course of pulmonary hypertension development: An experimental model of severe angioproliferative PAH was used. By week 5, the model produced a marked increase of right ventricle systolic pressure (RVSP; FIG. 1A), RV hypertrophy (FIG. 1B), and pulmonary artery remodeling (FIG. 2A and 2B). However, to evaluate the contribution of the early pathological events in PAH development and progression, the rats were also analyzed at two additional time points: week 1 (early stage of PAH) and week 2 (middle stage). The combination of SU5416 and hypoxia initiates a rapid increase in RVSP (FIG. 1A), which corresponded to the significant RV remodeling (FIG. 1B), and changes in RV function-increased RV contractility (FIG. 1C) and RV relaxation (FIG. 1D) as early as 1 week after PAH initiation. Interestingly, by week 2, the progression of PAH slowed down, and none of the physiological parameters analyzed were found to be significantly different between weeks 1 and 2. Nevertheless, at week 5 the disease accelerated again and resulted in pronounced changes that were found to be significantly different compared with both week 1 and week 2.

The histological evaluation of the lungs revealed that PAH induced a strong and progressive pulmonary vascular remodeling. However, the hypertrophy was found to be different for the small (<150 μM) and larger (≥150 μM) pulmonary arteries (PA). Thus, the small PAs continued to develop angioproliferative changes through the study, repeating the overall pattern of the disease development with quick progression at the early and late stages, but not at the middle stage. In contrast, the larger vessels responded only during the first week of the disease and were preserved during the rest of the study, thus confirming that the vasculopathy of small but not larger PAs is the primary contributor to PAH progression.

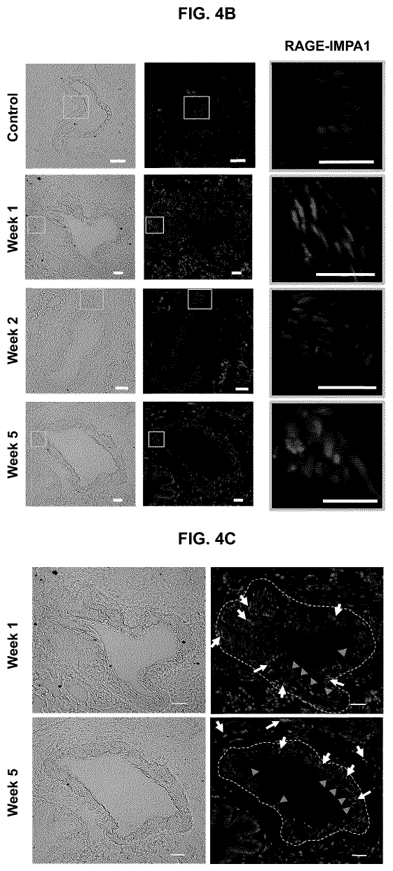

Apoptosis and RAGE activation in PAH: By measuring the level of cleaved caspase 3 in pulmonary tissue, the activation of early apoptosis in the lungs was confirmed (FIG. 3A). However, by week 2, apoptosis was resolved, which corresponded to the deceleration of the disease progression at this midpoint (FIG. 1A, 1B, 1C, and 1D and FIG. 2A and 2B). Finally, at the late stage of PAH (e.g., week 5), the apoptosis was evidenced again, suggesting that there is also a second delayed episode of PAH-induced damage that could contribute to the disease progression. To evaluate the particular source of apoptotic cells in PAH lungs, a TUNEL staining was performed. At the early stage (e.g., week 1), apoptosis was evident in endothelium and adventitia of pulmonary arteries (FIG. 3B), confirming that not only initial endothelial apoptosis but also damage that occurs in the adventitial layer is an important modulator of vascular remodeling. At the middle stage of PAH (e.g., week 2), the apoptosis positive cells were not found, suggesting that the initially apoptotic endothelial cells and fibroblasts have later transformed into apoptosis-resistant cells. By week 5, apoptosis manifested again and was visualized in all layers of the pulmonary vascular wall.

The factors released from dying cells are known to bind to RAGE and activate it. By measuring the level of RAGE interaction with its adaptor protein myeloid differentiation primary response 88 (MyD88), the level of RAGE activation was evaluated in pulmonary tissues of control and PAH rats. There was a strongly augmented interaction of RAGE and MyD88 during the early and late stages of PAH (FIG. 3C), confirming the activation of RAGE signaling specifically in weeks 1 and 5 of the disease.

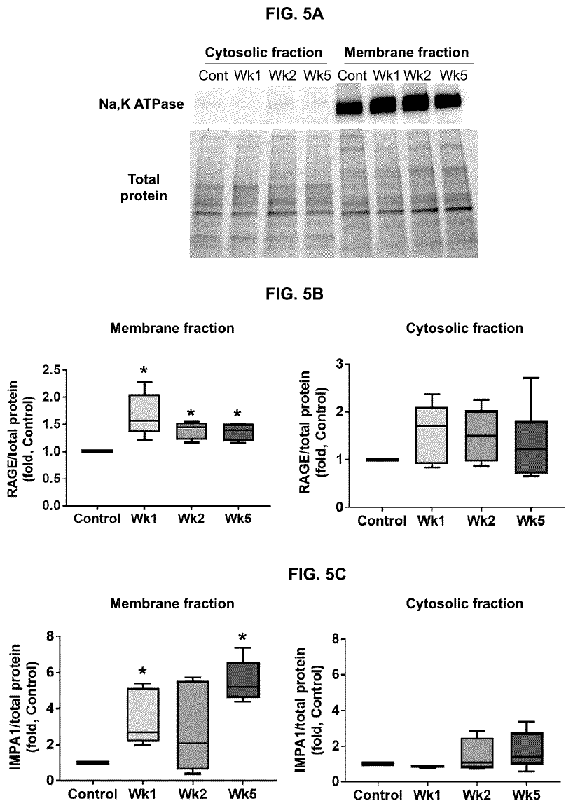

Pulmonary hypertension mediates the formation of the RAGE-IMPA1 complex: A mass spectrometry analysis was performed on the RAGE interactome to understand the particular downstream signaling induced by RAGE activation. IMPA1 was found to be one of RAGE interacting partners that bind to RAGE in PAH but not in control samples (FIG. 4A). Although the threshold was set to P>0, at least four peptides were found with a probability of >89%. The immunoprecipitation/immunoblotting analysis confirmed the absence of RAGE/IMPA1 interaction in control animals and revealed that the formation of this complex starts already at week 1 and is maintained throughout the duration of the disease (FIG. 4A).

To visualize the localization of complex RAGE-IMPA1 in pulmonary tissue, a Duolink proximity ligation assay (PLA) was performed. This technology generates an amplified signal only when the probes attached to each protein of interest are in close proximity (<40 nm). The PLA results have also confirmed that PAH initiates a direct interaction of RAGE and IMPA1 starting from week 1. The PLA signal was especially apparent in week 1 and week 5 (FIG. 4B). Importantly, the signal was visualized in the adventitial and medial layers of hypertrophied pulmonary arteries (FIG. 4C), supporting that RAGE and IMPA1 have an increased interaction in the pulmonary vascular wall, although the formation of the RAGE-IMPA1 complex was also seen in the pulmonary parenchyma.

To evaluate whether the interaction happens due to the translocation of IMPA1, known to be a cytosolic protein, on a plasma membrane, the distribution of RAGE and IMPA1 between cytosolic and membrane fraction was evaluated in control and diseased animals. By using the Na,K-ATPase as a plasma membrane marker, the efficiency of lung tissue fractionation was first validated in different animal sets (FIG. 5A). The membrane fraction had a strong Na,K-ATPase signal, whereas the cytosol was almost lacking the Na,K-ATPase signal. These validated samples were used to analyze the plasma membrane/cytosol distribution of RAGE, IMPA1, and other proteins. There was a significant accumulation of RAGE in the membrane fraction of samples from PAH rats (FIG. 5B) and a nonsignificant increase of RAGE in the cytosol fraction, possibly due to RAGE internalization and recycling. IMPA1 has also accumulated in the membrane but not in a cytosolic fraction (FIG. 5C). This increase was significant in week 1 and week 5, which corresponds with the profile of apoptosis-induced RAGE activation (FIG. 3C).

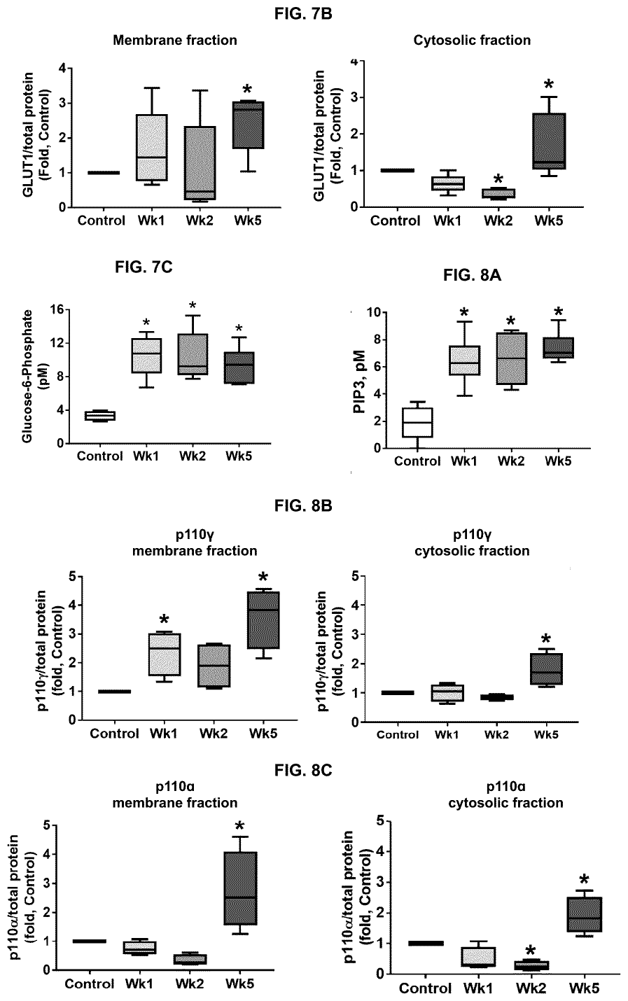

Computational docking was utilized to model the binding of RAGE region aa365-376 to the IMPA1 molecule. Docking of the RAGE-originated peptide 365-RRQRRGEERKAP-376 (SEQ ID NO: 36) to an IMPA1 structure utilizing molecular modeling algorithm confirmed a possible interaction between the intracellular positively charged arginine-rich region of RAGE and negatively charged COOH-terminus of IMPA1. Interestingly, this region of IMPA1 has a cavity that stretches into the active IMPA1 site, and the model predicted that one of the arginine residues from RAGE-peptide interacts with Mg²⁺ inside the active site of IMPA1 (FIG. 6A). However, the binding of RAGE peptide does not induce any sterical hindrance to the main entrance of the IMPA1 active site on the opposite side of the molecule (FIG. 6B). Analysis of the electrostatic map of IMPA1 showed additional, positively charged regions that the docking of IMPA1 to membranes due to electrostatic interactions. The negatively charged RAGE binding region of IMPA1 will be neutralized by positively charged RAGE residues (FIG. 6C), and this will additionally facilitate IMPA1-membrane interaction (FIG. 6D). Based on this analysis, the interaction between IMPA1 and RAGE includes 1) docking of the negatively charged COOH-terminus of IMPA1 to positively charged RAGE residues just below the intermembrane region of RAGE, 2) neutralization of the negative charge on the surface of IMPA1, and 3) binding the positively charged region of IMPA1 to the membrane (FIG. 6E).

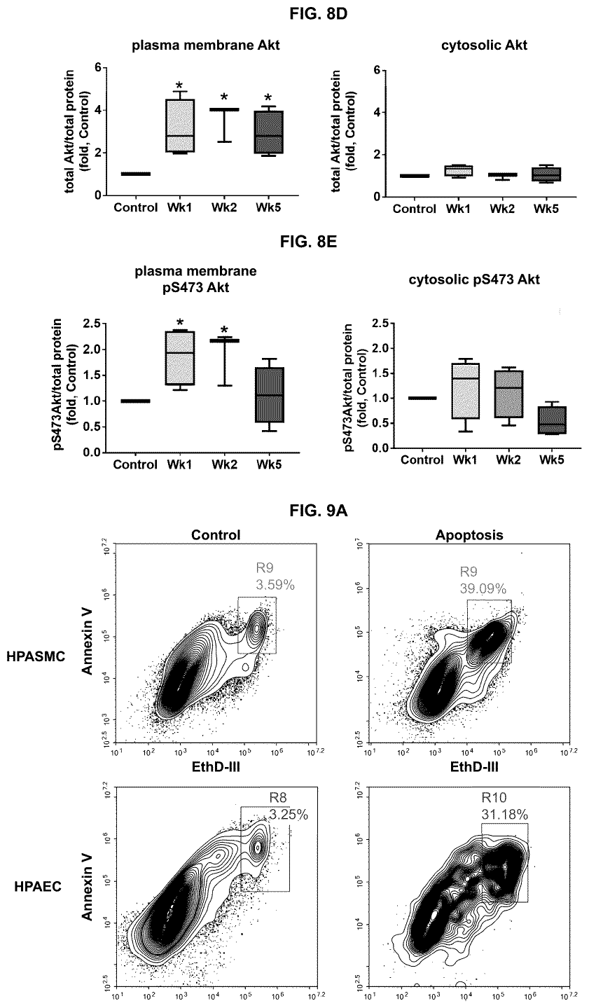

Pulmonary hypertension promotes an increase in pulmonary glucose uptake and metabolism: The shift of the pulmonary metabolism from mitochondrial oxidative phosphorylation to aerobic glycolysis is a hallmark of PAH that contributes to the hyperproliferative profile of pulmonary vascular cells. IMPA1 is known to be sensitive to increased levels of glucose metabolite G6P and catalyzes a critical step of G6P conversion to myo-inositol. Thus, the elevated glucose uptake and metabolism provide the circumstantial background for IMPA1 activation. To confirm that in the model, there was a metabolic shift and to evaluate the timing of the changes in metabolic profile, the membrane translocation of two main glucose transporters, GLUT1 and GLUT4, was measured in lung tissue from control and diseased animals. PAH induced a significant, almost four-fold increase in the amount of membrane GLUT4 at an early stage (week 1), which remained elevated through the study (FIG. 7A). This early membrane translocation of GLUT4 was not due to an increase in its expression, as the GLUT4 cytosolic levels were not significantly changed in week 1 or week 5 and only slightly increased in week 2. In contrast, GLUT1 showed a significant increase only by week 5 and was found to be upregulated in both membrane and cytosolic fractions (FIG. 7B). There was also a substantial accumulation of G6P in pulmonary hypertensive rats starting from week 1 (FIG. 7C) that confirmed the presence of a PAH-induced glycolytic shift in pulmonary tissue of these animals and a rational demand for IMPA1 activation.

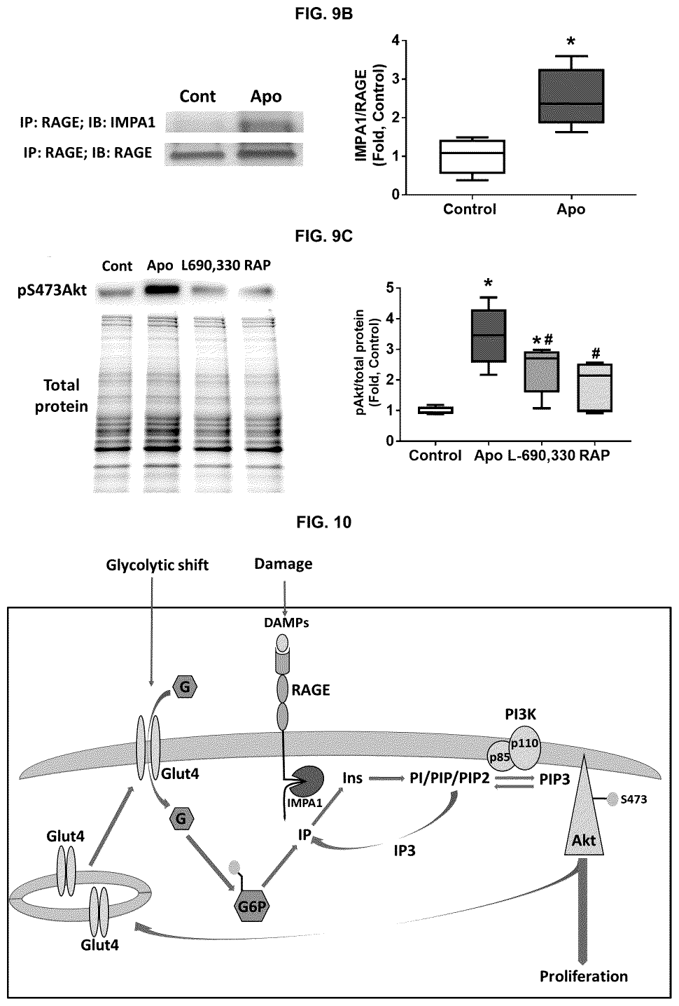

Activation of PIP₃-Akt axis is a characteristic of a developed and an incipient PAH: Myo-inositol generated by IMPA1 is a precursor of phosphatidylinositol 3,4,5-trisphosphate (PIP₃). PIP₃, in turn, ensures Akt binding to the membrane and allosterically activates Akt kinase by relieving an intramolecular autoinhibitory mechanism and permitting the binding of substrate. The formation of PIP₃ in the lipid fraction extracted from rat lungs is strongly increased upon PAH development (>3-fold greater in PAH rats compared with controls) starting from the earliest stage (week 1) (FIG. 8A). Because the synthesis of PIP₃ from phosphatidylinositol 4,5-bisphosphate (PIP₂) depends directly on the activity of phosphatidylinositol-3-kinase (PI3K), a time course of PI3K accumulation in the membrane fraction was also analyzed. PAH induced an increased translocation of PI3K catalytic subunit p110γ to the membrane in week 1 and week 5 (FIG. 8B). Importantly, the early accumulation of p110γ in the membrane fraction was not due to p110γ overexpression since the cytosolic portion of p110γ had not been altered until week 5 when it was mildly increased. Another catalytic subunit of PI3K, p110α, was also upregulated, but only by week 5, and found to be elevated in both membrane and cytosolic fractions (FIG. 8C). This finding suggests that p110γ may be the primary subunit responsible for the early synthesis of PIP₃.

As expected, the elevated levels of PIP₃ formation were associated with a sustained three-fold increase in Akt membrane translocation (FIG. 8D) and a twofold increase in membrane pSer473-Akt levels (FIG. 8E). As with RAGE, IMPA1, GLUT4, and p110γ, the cytosolic levels of total and phosphorylated Akt were not altered, supporting the specific role of membrane microdomains in proliferation signal transduction.

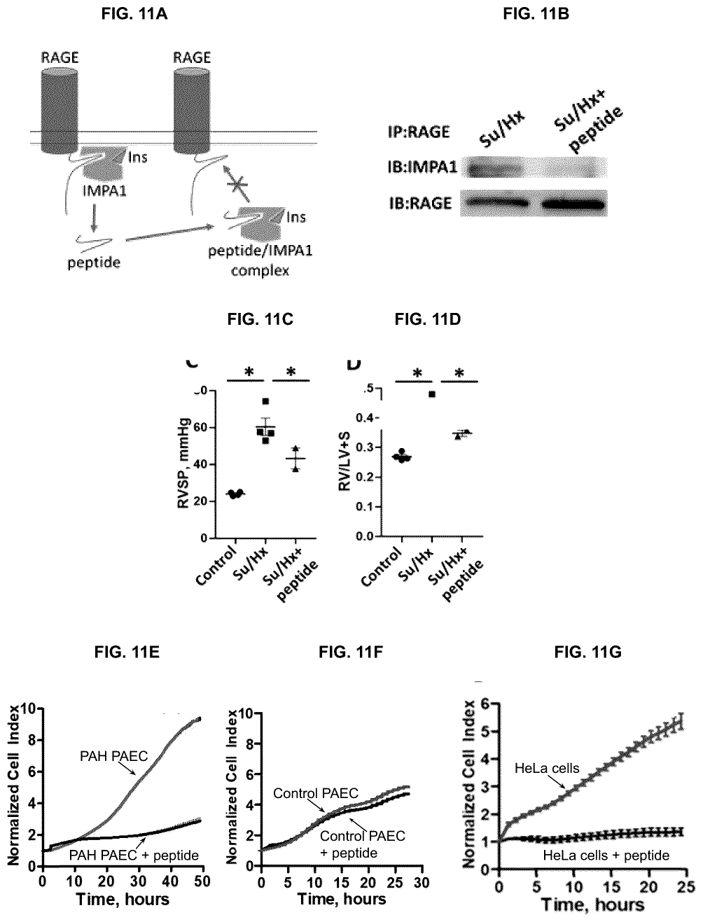

Formation of RAGE-IMPA1 complex and RAGE/IMPA-dependent activation of Akt in vitro: To validate the role of apoptosis in RAGE-IMPA1 interaction, in vitro experiments were performed in which apoptosis was initiated in pulmonary artery vascular cells by either keeping the cells in serum-free media (for HPAEC) or placing the cells in a low-glucose media (for HPASMC). Both treatments induced a significant apoptotic cell death that was confirmed by flow cytometry (FIG. 9A). The conditioned media collected from untreated and apoptotic cells stimulated the naïve HPASMC. Treatment with apoptotic media initiated interaction between RAGE and IMPA1 (FIG. 9B), confirming an important role of factors released from dying cells in the activation of RAGE and IMPA1. The same conditioned media were also used to evaluate the level of Akt activation in naïve HPASMC in the presence or absence of specific IMPA1 inhibitor L-690,330 and the RAGE antagonist RAP. Apoptotic Factors stimulated activation of Akt that was significantly attenuated in the presence of IMPA1 inhibitor and was even further reduced by RAGE antagonist (FIG. 9C). These results confirm an essential role of both proteins in the activation of Akt signaling.

The pathogenesis of PAH is complex. It requires multiple “hits” on the background of genetic predisposition to initiate and drive the disease progression. Although the key pathogenic events contributing to PAH have been identified, the clear interconnection between these events, as well as the mechanisms that initiate the disease transition from one stage to another, are not completely understood.

The present invention identifies that PAH initiates the interaction between RAGE and IMPA1 that is absent in healthy animals. The direct interaction between RAGE and IMPA1 may also interconnect a few pathological events confirmed to play an important role in PAH onset and progression. These include initial pulmonary vascular apoptosis, which stimulates DAMPs receptors such as RAGE, increased glucose uptake with subsequent glycolytic shift and activation of IMPA1, and reprogramming of survived vascular cells toward over proliferative cells.

### EMBODIMENTS

The following embodiments are intended to be illustrative only and not to be limiting in any way.

Embodiment 1: An antiproliferative peptide comprising a sequence at least 80% identical to ILWQRRQRRG (SEQ ID NO: 1) or ILWQRRQRRGEERKAP (SEQ ID NO: 2), wherein the peptide inhibits interaction between inositol monophosphatase 1 (IMPA1) and receptor for advanced glycation endproducts (RAGE).

Embodiment 2: The peptide of embodiment 1, wherein the peptide comprises at least four modifications.

Embodiment 3: The peptide of embodiment 1 or embodiment 2, wherein the peptide comprises at least one modification.

Embodiment 4: The peptide of any one of embodiments 1-3, wherein the modification is a substitution.

Embodiment 5: The peptide of embodiment 4, wherein the I amino acid is substituted with a V or an L amino acid.

Embodiment 6: The peptide of embodiment 4, wherein the L amino acid is substituted with an I or a V amino acid.

Embodiment 7: The peptide of embodiment 4, wherein the W amino acid is substituted with an F or a Y amino acid.

Embodiment 8: The peptide of embodiment 4, wherein at least one of the Q amino acids are substituted with an N amino acid.

Embodiment 9: The peptide of embodiment 4, wherein at least one of the R amino acids are substituted with a K amino acid.

Embodiment 10: The peptide of embodiment 4, wherein the G amino acid is substituted with an A amino acid.

Embodiment 11: The peptide of embodiment 4, wherein at least one of the E amino acids are substituted with a D amino acid.

Embodiment 12: The peptide of embodiment 4, wherein the K amino acid is substituted with an R amino acid.

Embodiment 13: The peptide of embodiment 4, wherein the A amino acid is substituted with a G amino acid.

Embodiment 14: The peptide of embodiment 4, wherein the P amino acid is substituted with an H amino acid.

Embodiment 15: The peptide of any one of embodiments 1-14, wherein the peptide is selected from a group consisting of SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, and SEQ ID NO: 38.

Embodiment 16: The peptide of any one of embodiments 1-15, wherein the amino acids are D-amino acids, L-amino acids, or a combination thereof.

Embodiment 17: The peptide of any one of embodiments 1-16, wherein an N-terminal or a C-terminal of the peptide is modified.

Embodiment 18: The peptide of embodiment 17, wherein the modification comprises adding a chemical moiety, a membrane crossing sequence, or a chemical group to the N-terminal or the C-terminal.

Embodiment 19: The peptide of embodiment 18, wherein the modification is a TAT sequence or a triphenylphosphonium group.

Embodiment 20: A method of treating a proliferation disease in a subject in need thereof, the method comprising, administering a therapeutically effective amount of a peptide according to any one of embodiments 1-19.

Embodiment 21: The method of embodiment 20, wherein the disease is pulmonary arterial hypertension (PAH).

Embodiment 22: The method of embodiment 20, wherein the disease is cancer.

Embodiment 23: A method of inhibiting an interaction between inositol monophosphatase 1 (IMPA1) and receptor for advanced glycation endproducts (RAGE) in vitro, the method comprising administering a peptide according to any one of embodiments 1-19 to a cell or an in vitro system.

Embodiment 24: A method of inhibiting an interaction between inositol monophosphatase 1 (IMPA1) and receptor for advanced glycation endproducts (RAGE) in a subject, the method comprising administering a peptide according to any one of embodiments 1-19 to the subject.

As used herein, the term “about” refers to plus or minus 10% of the referenced number.

Although there has been shown and described the preferred embodiment of the present invention, it will be readily apparent to those skilled in the art that modifications may be made thereto which do not exceed the scope of the appended claims. Therefore, the scope of the invention is only to be limited by the following claims. In some embodiments, the figures presented in this patent application are drawn to scale, including the angles, ratios of dimensions, etc. In some embodiments, the figures are representative only and the claims are not limited by the dimensions of the figures. In some embodiments, descriptions of the inventions described herein using the phrase “comprising” includes embodiments that could be described as “consisting essentially of” or “consisting of”, and as such the written description requirement for claiming one or more embodiments of the present invention using the phrase “consisting essentially of” or “consisting of” is met.

What is claimed is:

1. An antiproliferative peptide comprising a sequence at least 80% identical to ILWQRRQRRG (SEQ ID NO: 1) or ILWQRRQRRGEERKAP (SEQ ID NO: 2), wherein the peptide inhibits interaction between inositol monophosphatase 1 (IMPA1) and receptor for advanced glycation endproducts (RAGE).

2. The peptide of claim 1, wherein the peptide comprises at least four modifications.

3. The peptide of claim 1, wherein the peptide comprises at least one modification.

4. The peptide of claim 3, wherein the modification is a substitution.

5. The peptide of claim 4, wherein the I amino acid is substituted with a V or an L amino acid.

6. The peptide of claim 4, wherein the L amino acid is substituted with an I or a V amino acid.

7. The peptide of claim 4, wherein the W amino acid is substituted with an F or a Y amino acid.

8. The peptide of claim 4, wherein at least one of the Q amino acids are substituted with an N amino acid.

9. The peptide of claim 4, wherein at least one of the R amino acids are substituted with a K amino acid.

10. The peptide of claim 4, wherein the G amino acid is substituted with an A amino acid.

11. The peptide of claim 4, wherein at least one of the E amino acids are substituted with a D amino acid.

12. The peptide of claim 4, wherein the K amino acid is substituted with an R amino acid.

13. The peptide of claim 4, wherein the A amino acid is substituted with a G amino acid.

14. The peptide of claim 4, wherein the P amino acid is substituted with an H amino acid.

15. The peptide of claim 1, wherein the peptide is selected from a group consisting of SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, and SEQ ID NO: 38.

16. The peptide of claim 1, wherein the amino acids are D-amino acids, L-amino acids, or a combination thereof.

17. The peptide of claim 1, wherein an N-terminal or a C-terminal of the peptide is modified; wherein the modification comprises adding a chemical moiety, a membrane crossing sequence, or a chemical group to the N-terminal or the C-terminal.

18. A method of treating a proliferation disease in a subject in need thereof, the method comprising, administering a therapeutically effective amount of an antiproliferative peptide comprising a sequence at least 80% identical to ILWQRRQRRG (SEQ ID NO: 1) or ILWQRRQRRGEERKAP (SEQ ID NO: 2), wherein the peptide inhibits interaction between inositol monophosphatase 1 (IMPA1) and receptor for advanced glycation endproducts (RAGE).

19. The method of claim 18, wherein the disease is pulmonary arterial hypertension (PAH).

20. The method of claim 18, wherein the disease is cancer.
